(19) United States
(12) Patent Application Publication
(10) Pub. No.: US 2024/0099553 A1
(43) Pub. Date: Mar. 28, 2024

(54) ENDOLUMINAL DEVICE SYSTEM, CONTROL DEVICE, AND CONTROL METHOD

(71) Applicant: OLYMPUS MEDICAL SYSTEMS CORP., Tokyo (JP)

(72) Inventors: Noriaki YAMANAKA, Tokyo (JP); Shota SAWADA, Saitama (JP); Hiroshi ASHIBA, Saitama (JP); Saki ISHIZAWA, Tokyo (JP); Shun INOUE, Tokyo (JP)

(73) Assignee: OLYMPUS MEDICAL SYSTEMS CORP., Tokyo (JP)

(21) Appl. No.: 18/219,318

(22) Filed: Jul. 7, 2023

Related U.S. Application Data
(63) Continuation of application No. PCT/JP2022/007493, filed on Feb. 24, 2022.

(51) Int. Cl. A61B 1/00; A61B 1/005

(52) U.S. Cl. CPC A61B 1/00006; A61B 1/0057; A61B 2560/02; A61B 2560/04; A61B 2562/02

(57) ABSTRACT

Provided is an endoluminal device system including: an elongated and flexible endoluminal device; a wire that is disposed in the endoluminal device; a first sensor that detects tension of the wire; a second sensor that detects displacement of the wire; a driving device capable of pulling the wire; and a processor. The processor is configured to: cause the driving device to execute a relaxing operation for relaxing the wire, acquire the tension and the displacement of the wire during the relaxing operation, which are detected by the first sensor and the second sensor, respectively, and adjust a control parameter of the endoluminal device based on a rate of change in the tension with respect to the displacement.

### CROSS-REFERENCE TO RELATED APPLICATIONS

This is a continuation of International Application PCT/JP2022/007493, with an international filing date of Feb. 24, 2022, which is hereby incorporated by reference herein in its entirety.

### TECHNICAL FIELD

The present invention relates to an endoluminal device system, a control device, and a control method.

### BACKGROUND ART

Conventionally, there are known endoluminal devices such as manipulators or endoscopes that are inserted into a lumen from the mouth or anus to perform observation or treatment in the lumen (for example, see PTLs 1 to 3). The endoluminal device includes an elongated flexible portion, a movable portion connected to a distal end of the flexible portion, and a power transmission member such as a wire configured to drive the movable portion. In such an endoluminal device, there are problems that power transmission characteristics of the power transmission member change according to a shape of the flexible portion and thus operating characteristics of the movable portion change. In PTLs 1 to 3, a means for solving the problems is proposed.

Specifically, in PTLs 1 and 2, a sensor is used to detect a shape of the flexible portion, and the movable portion is controlled based on the detected shape. A magnetic sensor (UPD) arranged outside the endoluminal device is used in PTL 1, and a shape sensor to be inserted into the endoluminal device is used in PTL 2.

In PTL 3, a movable portion is previously operated in a plurality of operation patterns according to various shape patterns of a flexible portion, and control parameters are set in association with tension information of a power transmission member at that time. The tension information is a time integral value of tension, a rate of change, or a predetermined threshold. Then, the tension information is acquired in an intraoperative calibration operation, and control parameters based on the acquired tension information are acquired.

### CITATION LIST

### Patent Literature

{PTL 1} Japanese Unexamined Patent Application, Publication No. 2015-154814
{PTL 2} the Publication of Japanese Patent No. 6701232
{PTL 3} the Publication of Japanese Patent No. 6278747

### SUMMARY OF INVENTION

A first aspect of the present invention provides an endoluminal device system including: an elongate and flexible endoluminal device; a flexible first wire that is disposed in the endoluminal device along a longitudinal direction of the endoluminal device; a first sensor that detects tension of the first wire; a second sensor that detects displacement of the first wire; a driving device capable of pulling the first wire; and at least one processor, wherein the at least one processor is configured to: cause the driving device to execute a relaxing operation for relaxing the first wire, acquire the tension of the first wire during the relaxing operation detected by the first sensor, acquire the displacement of the first wire during the relaxing operation detected by the second sensor, and adjust a control parameter of the endoluminal device based on a rate of change in the tension with respect to the displacement during the relaxing operation.

A second aspect of the present invention provides a control device for an endoluminal device system, the endoluminal device system including: an elongated and flexible endoluminal device; a flexible first wire that is disposed in the endoluminal device along a longitudinal direction of the endoluminal device; a first sensor that detects tension of the first wire; a second sensor that detects displacement of the first wire; and a driving device capable of pulling the first wire, wherein the control device comprises at least one processor, and wherein the at least one processor is configured to: cause the driving device to execute a relaxing operation for relaxing the first wire, acquire the tension of the first wire during the relaxing operation detected by the first sensor, acquire the displacement of the first wire during the relaxing operation detected by the second sensor, and adjust a control parameter of the endoluminal device based on a rate of change in the tension with respect to the displacement during the relaxing operation.

A third aspect of the present invention provides a control method for an endoluminal device, the endoluminal device having a flexible first wire disposed therein along a longitudinal direction of the endoluminal device, wherein the control method includes: executing a relaxing operation for relaxing the first wire; acquiring tension of the first wire during the relaxing operation; acquiring displacement of the first wire during the relaxing operation; and adjusting a control parameter of the endoluminal device based on a rate of change in the tension with respect to the displacement during the relaxing operation.

### BRIEF DESCRIPTION OF DRAWINGS

FIG. 1 is an external view showing an entire configuration of an endoluminal device system according to a first embodiment.

FIG. 2 is a schematic configuration diagram of the endoluminal device system according to the first embodiment.

FIG. 3 is a flowchart of a control method according to the first embodiment.

FIG. 4 is a diagram showing shapes of wires with loops formed at a distal portion (upper stage), a center (middle stage), and a proximal portion (lower stage).

FIG. 5 is a graph showing a change in tension over time during a relaxing operation at the proximal end and the distal end of each of the wires having each of the shapes shown in FIG. 4.

FIG. 6A is a diagram showing an example of a distribution of tension from the proximal end to the distal end of the wire.

FIG. 6B is a diagram showing another example of a distribution of tension from the proximal end to the distal end of the wire.

FIG. 7 is a diagram showing an example of a shape of a wire.

FIG. 8 is a graph showing a relationship between displacement and tension of a wire in a relaxing operation.

FIG. 9 is a flowchart of a modified example of the control method shown in FIG. 3.

FIG. 10 is a schematic configuration diagram of another modified example of the endoluminal device system shown in FIG. 2.

FIG. 11 is a flowchart of another modified example of the control method shown in FIG. 3.

FIG. 12 is a diagram illustrating a method of estimating a shape of a flexible portion.

FIG. 13 is a flowchart showing a procedure for estimating the shape of the flexible portion.

FIG. 14 is a schematic configuration diagram of an endoluminal device system according to a second embodiment.

FIG. 15 is another schematic configuration diagram of an endoluminal device system according to the second embodiment.

FIG. 16 is a schematic configuration diagram of another modified example of the endoluminal device system shown in FIG. 14.

FIG. 17 is a graph showing an example of spring characteristics of a measuring wire.

FIG. 18 is a schematic configuration diagram of an endoluminal device system according to a third embodiment.

FIG. 19 is a schematic configuration diagram of an endoluminal device system according to another embodiment.

FIG. 20 is a graph showing a relationship between the amount of pulling of the proximal end of the wire and tension at the proximal end and tension at the distal end of the wire.

FIG. 21A is a flowchart of a control method according to one aspect of a fourth embodiment.

FIG. 21B is a flowchart of a control method according to another aspect of the fourth embodiment.

FIG. 21C is a flowchart of a control method according to further another aspect of the fourth embodiment.

FIG. 22 is a schematic configuration diagram of an endoluminal device system according to a fifth embodiment.

FIG. 23 is a flowchart of a control method according to the fifth embodiment.

FIG. 24 is a diagram illustrating a method of estimating a shape of a flexible portion.

FIG. 25 is a diagram showing a model of an endoluminal device.

FIG. 26 is a graph showing a relationship between the amount of pulling of a proximal end of a wire and tension at the proximal end of the wire and illustrating three states of the wire.

FIG. 27 is a flowchart of step S4 in a control method according to a sixth embodiment.

FIG. 28A is a diagram showing a conventional spring model of a wire.

FIG. 28B is a diagram showing an improved spring model of a wire.

FIG. 29 is a diagram illustrating a step time of calculation executed by a processor.

FIG. 30 is a diagram showing a relationship between a wire state and a spring constant.

### DESCRIPTION OF EMBODIMENTS

### First Embodiment

An endoluminal device system, a control device, and a control method according to a first embodiment of the present invention will be described with reference to the drawings.

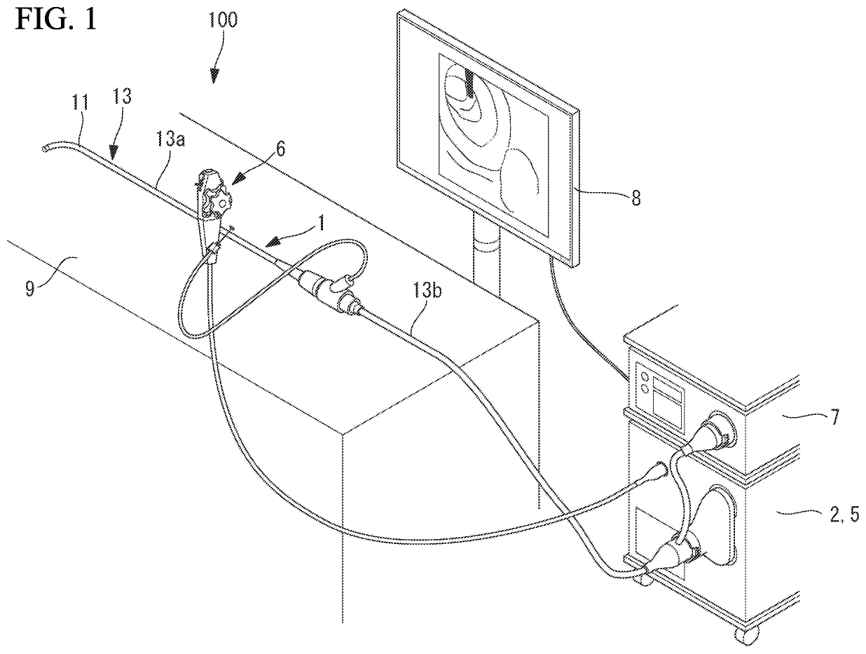

FIG. 1 shows an external view of one configuration example of an endoluminal device system 100 according to the present embodiment. In the endoluminal device system 100, a control device 5 controls a driving device 2 based on a manipulation input to a manipulation device 6, whereby a movable portion 11 of the endoluminal device 1 is electrically driven.

The endoluminal device 1 is an elongated and flexible device, for example, an endoscope or a manipulator, which is inserted into a body cavity through an opening such as a mouth or anus for observation or treatment within the body cavity. FIG. 1 shows a system 100 as an example in which the endoluminal device 1 is an endoscope. Reference numeral 7 denotes an image processor for processing an endoscopic image acquired by the endoscope 1, and reference numeral 8 denotes a display device for displaying an endoscopic image. The manipulation device 6 is a manipulation unit such as a rotatable knob provided on the endoluminal device 1.

The driving device 2 incorporating a motor and the like is placed aside an operating table 9, as an example. Therefore, a flexible portion 13 of the electrically driven endoluminal device 1 includes, as well as an elongated intracorporeal portion 13a that is inserted into a body, an elongated extracorporeal portion 13b that is placed outside the body and connects a proximal end of the intracorporeal portion 13a to the driving device 2. Such a flexible portion 13 having flexibility is bendable in various shapes. In particular, since the shape of the extracorporeal portion 13b is determined by an arrangement of the driving device 2 and the operating table 9, the shape varies largely. Power transmission characteristics of a wire 12 in the endoluminal device 1 change depending on the shape of the flexible portion 13, and thus responsiveness of the movable portion 11 to the manipulation input to the manipulation device 6 can also change. As will be described below, the endoluminal device system 100 has a function of controlling the endoluminal device 1 such that the responsiveness of the movable portion 11 is constant by controlling the driving device 2 depending on the shape of the flexible portion 13.

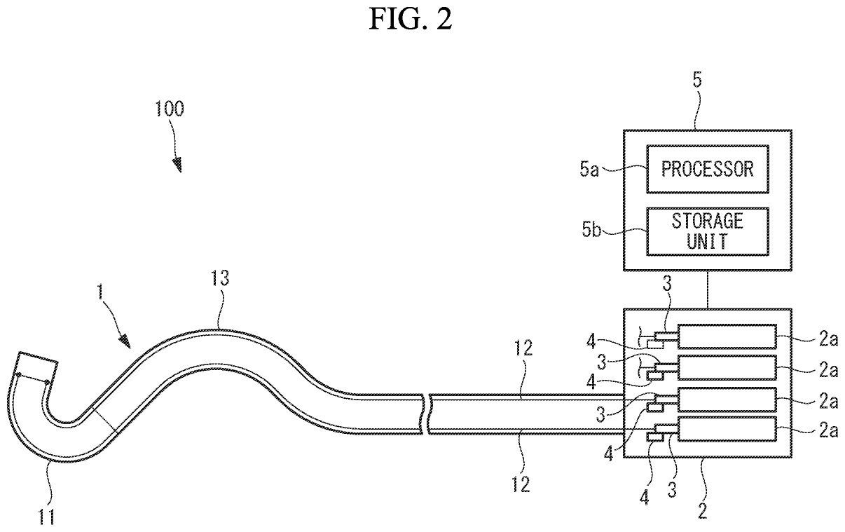

FIG. 2 shows a schematic configuration of the endoluminal device. As shown in FIG. 2, the endoluminal device system 100 includes an endoluminal device 1 having a movable portion 11, one or more flexible wires (first wires) 12 provided in the endoluminal device 1, a driving device 2 that drives the movable portion 11 by pulling and relaxing the wires 12, one or more tension sensors (first sensors) 3 that detect tension of each of the wires 12, one or more displacement sensors (second sensors) 4 that detect displacement of each of the wires 12, and a control device 5 that controls the driving device 2.

The endoluminal device 1 includes an elongated and flexible portion 13. A proximal end of the flexible portion 13 is connected to the driving device 2.

The movable portion 11 is connected to a distal end of the flexible portion 13. In the present embodiment, the movable portion 11 is a bending portion that is bendable in a direction intersecting a longitudinal direction of the flexible portion 13.

The wire 12 has a function of transmitting the power given from the driving device 2 from the proximal end thereof to the distal end thereof to allow the movable portion 11 to move, and a function of acquiring shape information of the flexible portion 13. Hereinafter, the wire having the function of transmitting the power given from the driving device 2 from the proximal end to the distal end to allow the movable portion 11 to move is referred to as a driving wire, and the wire having the function of acquiring shape information of the flexible portion 13 is referred to as a measuring wire. For example, four wires 12 are provided to bend the bending portion 11 upward, downward, left, and right, respectively. In the present embodiment, each of these four wires 12 has both of a function as a driving wire and a function as a measuring wire.

The wire 12 is disposed in the flexible portion 13 along the longitudinal direction of the flexible portion 13, the distal end of the wire 12 is fixed to a distal end of the bending portion 11, and the proximal end of the wire 12 is connected to the driving device 2. The flexible wire 12 extending over the entire length of the flexible portion 13 in this way bends into the same shape as the flexible portion 13 as the flexible portion 13 bends.

The driving device 2 includes one or more actuators 2a that drive the wire 12 by retracting and advancing the proximal end of the wire 12 in the longitudinal direction of the wire 12. The actuator 2a pulls the wire 12 by retracting the proximal end of the wire 12, and relaxes the wire 12 by advancing the proximal end of the wire 12. The driving device 2 actuates the actuator 2a according to a control signal (which will be described below) from the control device 5 to bend the bending portion 11.

The tension sensor 3 is provided at the proximal end portion of the wire 12 and detects tension at the proximal end portion of the wire 12.

The displacement sensor 4 detects displacement of the proximal end portion in the longitudinal direction of the wire 12. For example, the displacement sensor 4 is an angle sensor that is provided in the actuator 2a and detects a rotation angle of the motor of the actuator 2a as displacement of the wire 12. A position of the proximal end portion of the wire 12 is an origin when the tension of the wire 12 is zero, and the displacement of the proximal end portion of the wire 12 detected by the displacement sensor 4 is a position of the proximal end of the wire 12 with respect to the origin. Therefore, the displacement increases with pulling of the wire 12, and decreases with relaxing of the wire 12.

The control device 5 includes at least one processor 5a such as a central processing unit, and a storage unit 5b.

The processor 5a receives a manipulation signal based on the input manipulation from the manipulation device 6, generates a control signal based on the manipulation signal, and transmits the control signal to the driving device 2. Thereby, the processor 5a controls the driving device 2 and thus controls the bending portion 11 of the endoluminal device 1.

The storage unit 5b includes a memory such as an RAM and a nonvolatile recording medium such as a ROM or an HDD. The recording medium is a computer-readable non-transitory recording medium. The recording medium stores a control program for causing the processor 5a to execute a control method for controlling the endoluminal device 1 according to the shape of the flexible portion 13.

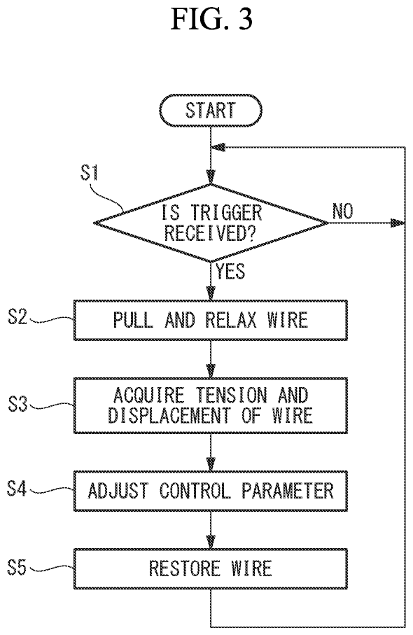

Next, a control method of the endoluminal device 1 to be executed by the control device 5 will be described with reference to FIG. 3.

As shown in FIG. 3, the control method includes step S1 of receiving a trigger, step S2 of executing a relaxing operation to relax the wire 12, step S3 of acquiring the tension and the displacement of the wire 12 during the relaxing operation detected by the sensors 3 and 4, respectively, step S4 of adjusting control parameters of the endoluminal device 1 based on the tension and the displacement during the relaxing operation, and step S5 of restoring the wire 12 to an initial state.

An operator inputs the trigger to the control device 5 using an input device (not shown), for example. The operator can input the trigger to the control device 5 at any desired timing when the operator wants to adjust the control parameters. For example, the operator inputs the trigger immediately before performing work such as treatment that requires an accuracy operation of the bending portion 11 after inserting the flexible portion 13 into the body cavity.

The processor 5a executes steps S2 to S5 in response to receiving the input trigger (YES in step S1).

Before step S2, the wire 12 and the bending portion 11 are arranged in a predetermined initial state. The initial state is, for example, a state in which predetermined initial tension is applied to all of the wires 12 and the bending portion 11 is arranged in a straight line.

In step S2, the processor 5a causes the driving device 2 to execute a pulling operation to pull the wire 12, and then causes the driving device 2 to execute a relaxing operation to relax the wire 12 being pulled. The wire 12 to be pulled and relaxed is any one wire 12, and the wire 12 paired with such a wire 12 is driven by the same amount, for example, in an opposite direction such that tension is not applied to the bending portion 11.

In the pulling operation, the processor 5a causes, for example, the driving device 2 to pull the wire 12 by a predetermined amount such that the wire 12 is applied with predetermined target tension. The predetermined target tension is within a range that does not hinder the bending of the flexible portion 13 along the shape of the body cavity, and accordingly, the wire 12 after the pulling operation is in a relaxed state to allow the bending of the flexible portion 13 along the shape of the body cavity.

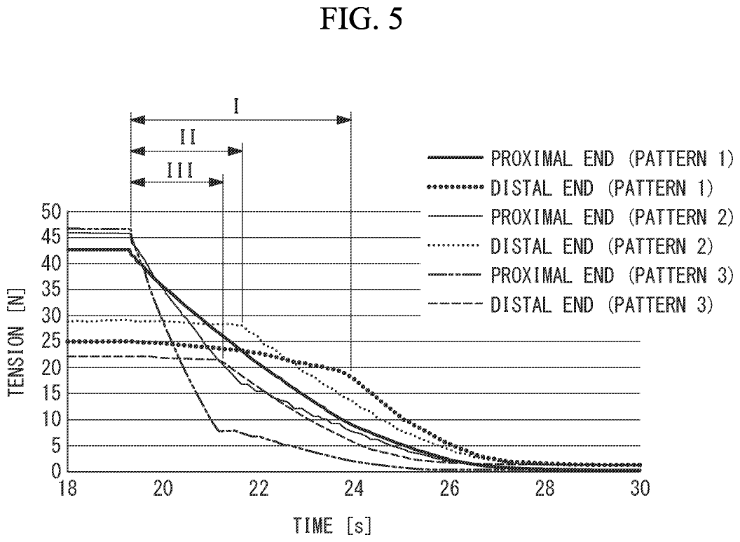

During the relaxing operation, the displacement of the proximal end of the wire 12 decreases over time and the tension at the proximal end of the wire 12 is reduced over time (for example, see FIG. 5). During the relaxing operation, the changing tension and displacement of the wire 12 are continuously detected by the sensors 3 and 4 at predetermined sampling periods.

Here, as the displacement of the proximal end of the wire 12 decreases, a region where tension decreases spreads from the proximal end to the distal end of the wire 12. In other words, during the relaxing operation, a change in tension (a decrease in tension) is transferred sequentially from the proximal end to the distal end of the wire 12. The processor 5a causes the driving device 2 to execute the relaxing operation at least until the change in tension is transferred to the distal end of the wire 12.

For example, the processor 5a may sequentially acquire the current tension or displacement from the sensors 3 and 4 during the relaxing operation, determine based on the current tension or displacement that the overall relaxing of the wire 12 is completed, and end the relaxing operation when the relaxing is completed. When the current tension is equal to or less than a predetermined value or when the current displacement is equal to or less than a predetermined value, the relaxing is determined to be completed.

When the change in tension reaches the distal end of the wire 12, there may appear a change point where a rate of change in tension changes. The processor 5a may end the relaxing operation when the change point of the tension is detected.

In step S3, the processor 5a causes the storage unit 5b to store the tension and the displacement during the relaxing operation detected by the sensors 3 and 4 in time series. Thereby, the processor 5a acquires tension data and displacement data during the relaxing operation.

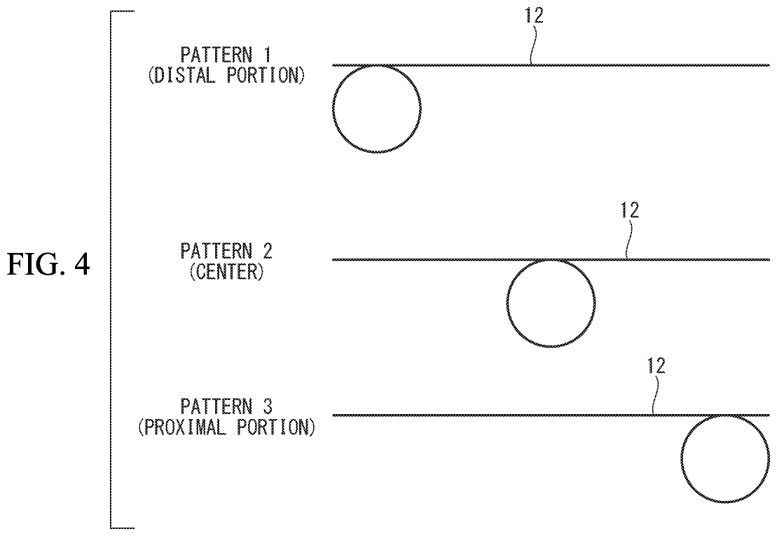

FIG. 5 shows a change in tension over time during a relaxing operation at the proximal end and the distal end of the wire 12 in three shaped patterns 1, 2, and 3 of the flexible portion 13 with loops formed at a distal end, a center, and a proximal end, respectively, as shown in FIG. 4. As shown in FIG. 5, the change in tension in periods I, II, and III until the change in tension reaches the distal end of the wire 12 differs depending on the shape of the flexible portion 13. In other words, the change in tension during the relaxing operation includes the shape information of the flexible portion 13.

Next, in step S4, the processor 5a uses the tension data and the displacement data acquired in step S3 to calculate the rate of change in tension with respect to the displacement during the relaxing operation, and adjusts the control parameters based on the rate of change. The rate of change in tension is a rate of change in tension at each displacement from the maximum tension at the start of relaxing operation.

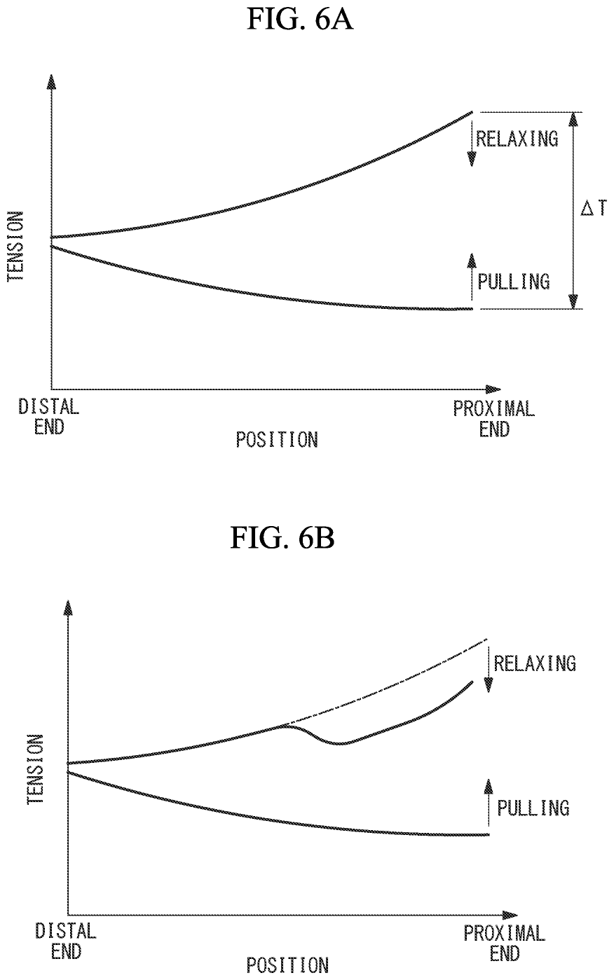

The control parameters are the amount of pulling and the amount of relaxing of the wire 12 to transmit the change in tension to the distal end of the wire 12. More specifically, the control parameter is the amount of displacement of the proximal end of the wire 12 in the periods I, II, and III shown in FIG. 5. In other words, the control parameter is the amount of displacement of the proximal end of the wire 12 equivalent to a change in tension at the proximal end required to transmit the change ΔT in tension to the distal end of the wire 12, as shown in FIG. 6A.

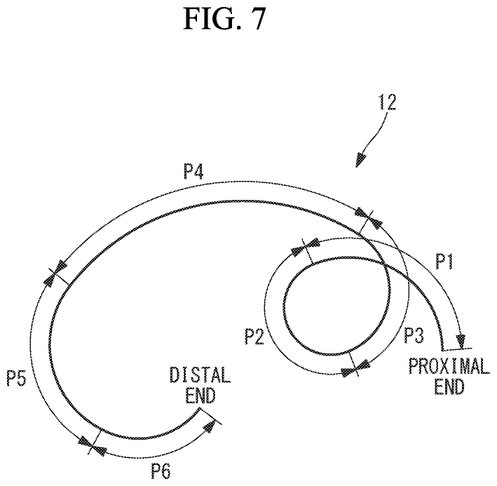

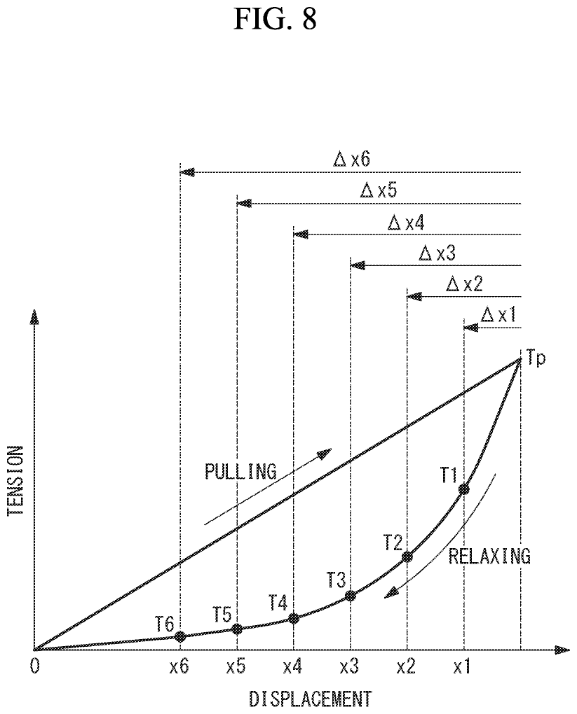

FIGS. 7 and 8 illustrate the shape of the wire 12 and a relationship between the displacement and the tension of the wire 12 during the relaxing operation, respectively. Since the change in tension at the proximal end of the wire 12 is transferred sequentially from the proximal end to the distal end of the wire 12, the change in tension at each displacement Δxi (i=1, 2, . . . ) of the proximal end of the wire 12 includes shape information of a corresponding portion Pi of the wire 12.

For example, when the displacement of the proximal end of the wire 12 is reduced by Δx1, the change in tension is transferred to a portion P1 of the wire 12. Therefore, the change in tension at the displacement Δx1 include shape information of the portion P1. When the displacement of the proximal end of the wire 12 is further reduced by Δx2, the change in tension is transferred to a portion P2 of the wire 12. Therefore, the change in tension at the displacement Δx2 includes shape information of the portions P1 and P2.

Therefore, a shape of a portion Pi can be estimated from a rate of change Ti/Tp in tension with respect to the displacement xi in order from the proximal end of the wire 12, and the control parameter corresponding to the overall shape of the flexible portion 13 can be determined based on such a rate of change Ti/Tp.

In order to accurately estimate the shape of the flexible portion 13 from the change in tension during the relaxing operation, as shown in FIG. 6A, it is preferable that the tension of the wire 12 disposed along the flexible portion 13 monotonically decreases or increases from the proximal end to the distal end. Thus, the processor 5a continues to gradually reduce the tension at the proximal end by continuing to advance the proximal end of the wire 12 until the change in tension is transferred to the distal end during the relaxing operation.

For example, when the relaxing of the wire 12 is completed and pulled again before the change in tension reaches the distal end, the tension of the wire 12 does not monotonically decrease from the proximal end to the distal end, as shown in FIG. 6B. In this case, a relationship between displacement and tension when the maximum displacement is updated is extracted.

After completing the relaxing operation, in step S5, the processor 5a causes the driving device 2 to execute an initialization operation for returning the tension of the wire 12 to the initial tension. Thus, the wire 12 and the bending portion 11 return to the initial state.

After step S5, the processor 5a generates a control signal based on the manipulation signal from the manipulation device 6 and the control parameters adjusted in step S4, and transmits the control signal to the driving device 2. Thus, regardless of the shape of the flexible portion 13, the bending portion 11 can be controlled with good responsiveness.

As described above, according to the present embodiment, the change in tension of the wire 12 during the relaxing operation differs depending on the bending shape of the flexible portion 13, and the shape of the flexible portion 13 corresponding to the change in tension of the wire 12 is unique. Therefore, it is possible to set appropriate control parameters according to the shape of the flexible portion 13 based on the change in tension of the wire 12.

Further, with a simple configuration using the wire 12 and the sensors 3 and 4, it is possible to acquire the required data and set the control parameters.

In addition, the wire 12 has a function as a driving wire and a function as a measuring wire. Thus, there is no need to mount a separate measuring wire for acquiring shape information, or to add a new component for measurement to the flexible portion 13. Therefore, it is possible to reduce a diameter of the flexible portion 13.

In addition, the tension data and the displacement data may be acquired in the operation in which the flexible portion 13 is inserted into the body cavity and the bending portion 11 being bent returns to a straight shape. In this case, no manipulation is required to acquire the data, and data necessary for adjusting the control parameters can be acquired without increasing labor of the operator.

In addition, the data is acquired and the control parameters are adjusted immediately before the work that requires an accuracy operation of the bending portion 11, whereby optimum control parameters can be reliably set according to the shape of the flexible portion 13 at that time, and the bending portion 11 can be accurately moved according to the manipulation input to the manipulation device 6.

In the present embodiment, the processor 5a may cause the driving device 2 to execute pulling and relaxing operations without movement of the bending portion 11 by selecting the wire 12 to be pulled and relaxed.

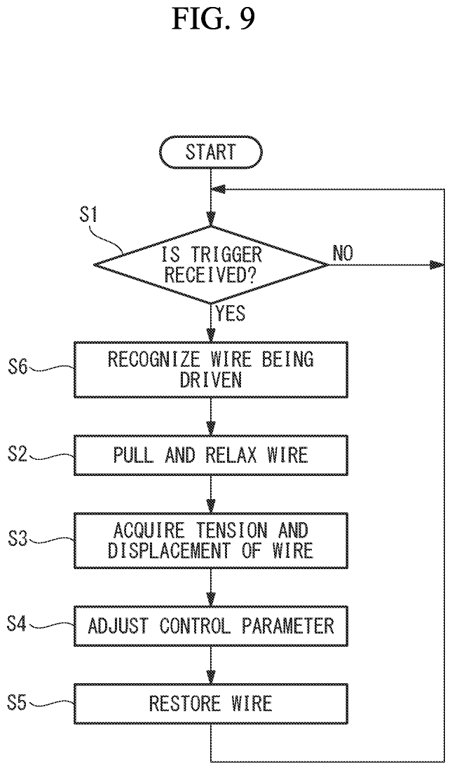

Specifically, as shown in FIG. 9, before executing the pulling operation and the relaxing operation in step S2, the processor 5a recognizes, from the plurality of wires 12, a wire 12 which is being driven and pulled for bending the bending portion 11, based on the manipulation signal from the manipulation device 6 (step S6). The wire 12 being driven may be recognized, for example, based on the control signal transmitted from the control device 5 to the driving device 2 or the tension detected by the tension sensor 3.

In step S2 of a first example of the control method in FIG. 9, the processor 5a selects the wire 12 being driven and one wire 12 opposing the wire 12 being driven. The opposing wire 12 is a wire for bending the bending portion 11 in a direction opposite to the bending direction of the bending portion 11 by the wire 12 being driven. For example, when the wire 12 being driven is an upward bending wire 12, the opposing wire 12 is a downward bending wire 12.

Next, the processor 5a controls the driving device 2 to simultaneously pull and relax the two selected wires 12 in the pulling operation and the relaxing operation. The relaxing operation at this time is executed until the tension of the wire 12 being driven returns to the tension before the pulling operation. The processor 5a acquires tension data and displacement data of at least one of the two wires 12 during the relaxing operation, and adjusts the control parameters based on the acquired data.

With such a configuration, the moment applied to the bending portion 11 is balanced during the pulling operation and the relaxing operation, and thus a bending direction and a bending angle of the bending portion 11 are kept constant. Therefore, it is possible to acquire the tension data and the displacement data without causing the operation of the bending portion 11.

In a second example of the control method in FIG. 9, the processor 5a selects any one wire 12 other than the wire 12 being driven, that is, selects one from the wires 12 that are not being driven. For example, one is selected from wires other than the wire 12 being driven and the opposing wire 12. Specifically, when the wire 12 being driven is an upward or downward bending wire, a leftward or rightward bending wire 12 is selected. When the wire 12 being driven is a leftward or rightward bending wire, the upward or downward bending wire 12 is selected.

Next, the processor 5a controls the driving device 2 to pull and relax the one selected wire 12 and the wire 12 paired with the selected wire 12 in the pulling operation and the relaxing operation.

With such a configuration, it is also possible to acquire the tension data and the displacement data without causing the operation of the bending portion 11 during the pulling operation and the relaxing operation.

In the present embodiment, the movable portion 11 is the bending portion, but the movable portion 11 is not limited thereto, and may have other forms. For example, the movable portion 11 may be a joint or may be an end effector having a grasping function.

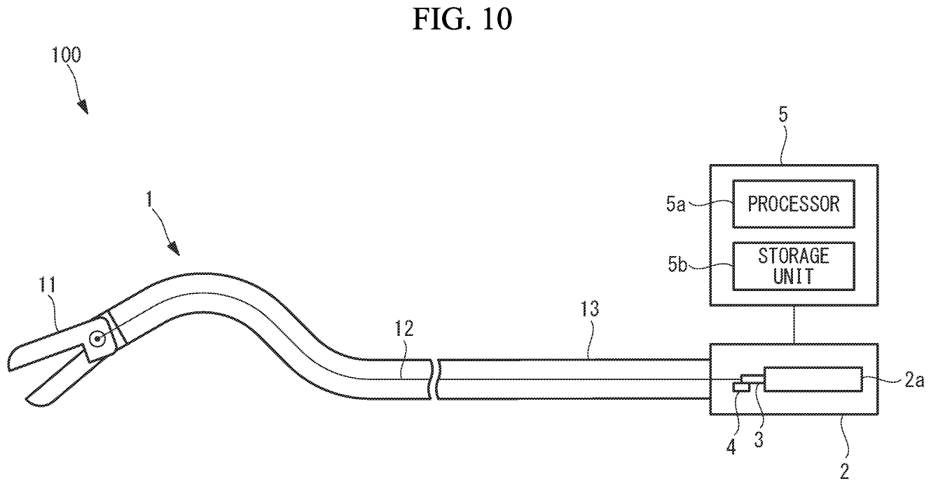

FIG. 10 shows an endoluminal device 1 including an end effector as the movable portion 11. In this example, the end effector 11 has a pair of jaws that open and close with each other, and the pair of jaws are opened and closed as the wire 12 is driven.

In the present embodiment, the processor 5a executes the control method in response to the trigger input by the operator, but may automatically execute the control method.

For example, the processor 5a may repeatedly execute steps S2 to S5 at predetermined time interval. In this case, the acquisition of data and the adjustment of control parameters are repeatedly and automatically executed without the input of the trigger from the operator.

For example, the shape of the flexible portion 13 continuously changes in the process of inserting the flexible portion 13 into the body cavity. In such a scene, the control parameters are sequentially updated according to the change in the shape of the flexible portion 13, and thus the responsiveness of the movable portion 11 can be kept good.

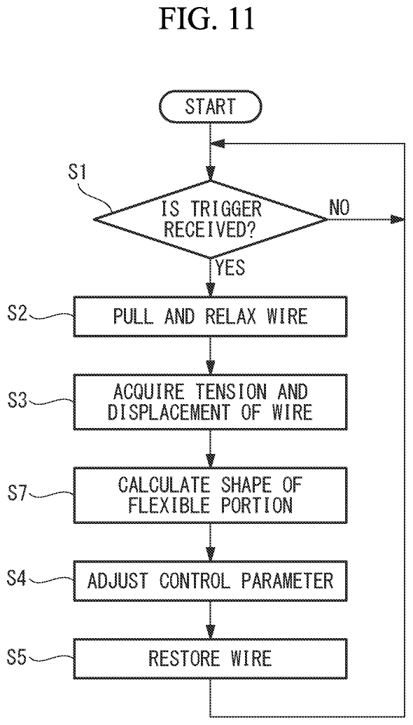

In the present embodiment, as shown in FIG. 11, the processor 5a may estimate the shape of the flexible portion 13 based on the tension data and the displacement data (step S7). In this case, in step S4, the processor 5a may adjust the control parameters based on the rate of change in tension as described above, or may adjust the control parameters based on the shape of the flexible portion 13.

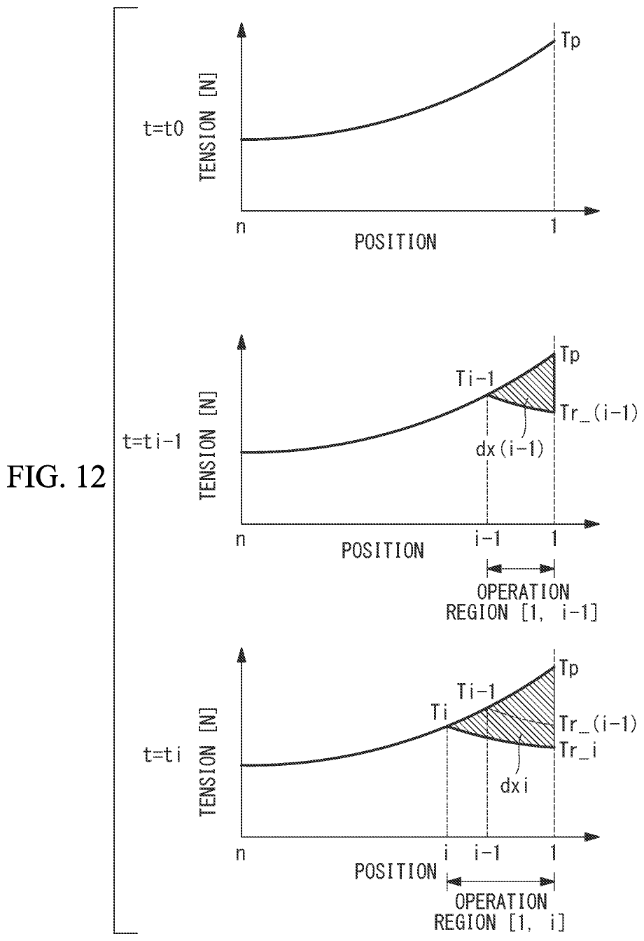

FIG. 12 illustrates a method of estimating the shape of the flexible portion 13 from the tension and the displacement of the wire 12, and shows distribution of tension from the proximal end to the distal end of the wire 12. By this method, shape information such as a bending radius, a bending angle, and a length along a main axis of the flexible portion 13 can be calculated.

As shown in FIG. 12, it is considered that the wire 12 is composed of n elements i (i=1, 2, . . . , n) in order from the proximal end to the distal end. Element 1 includes the proximal end of the wire 12, and element n includes the distal end of the wire 12.

In an initial state of t=t0, the tension of the wire 12 decreases exponentially from the proximal end to the distal end due to the effect of friction acting on the wire 12. As described above, in the process in which the wire 12 relaxes due to the displacement (advancement) of the proximal end of the wire 12, the change in tension (decrease in tension) is transferred sequentially from the proximal end to the distal end.

At t=ti−1, the tension at the proximal end drops from the initial maximum tension Tp to tension Tr_(i−1), and the change in tension is transferred to element i−1. In other words, the tension decreases in an operation region {1:i−1}, and the elongation of elements 1, 2, . . . , i−1 within the operation region {1:i−1} changes (decreases).

Similarly, at t=ti, the tension at the proximal end drops from the initial maximum tension Tp to tension Tr_i, and the change in tension is transferred to the element i.

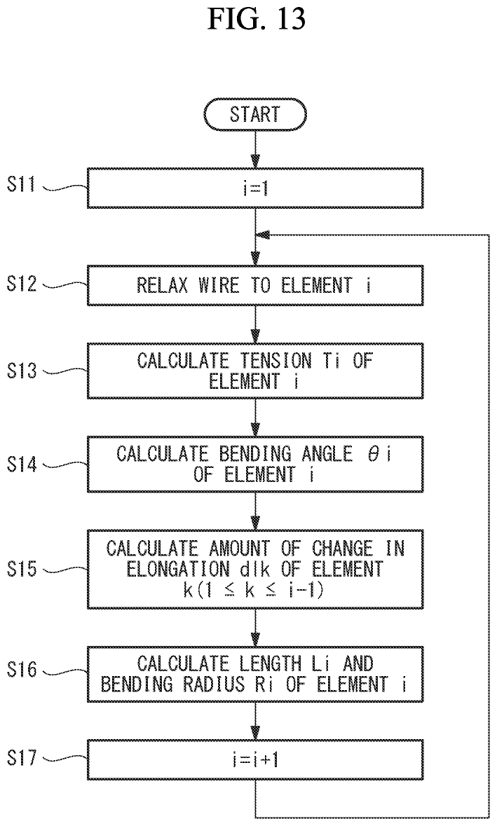

FIG. 13 shows a calculation procedure of the shape of the wire 12 (that is, the shape of the flexible portion 13). As shown in FIG. 13, by repeating steps S12 to S16 while incrementing i (steps S11, S17), a bending angle θi, a length Li, and a bending radius Ri of an element i are calculated in order from the proximal end. The calculated bending angle θi, length Li, and bending radius Ri are stored in the storage unit 5b and used to calculate a bending angle θi+1, a length Li+1, and a bending radius Rill of the next element i+1. Steps S12 to S16 are repeated until the sum of the lengths Li reaches the total length of the wire 12, thereby obtaining shapes θi, Li, and Ri of all element i.

In step S12, as the displacement of the proximal end of the wire 12 is reduced by dxi, the tension at the proximal end decreases from Tr_(i−1) to Tr_i, and the wire 12 relaxes to the element i, that is, the operation region extends to the element i.

Next, in step S13, tension Ti of the element i is calculated.

The maximum tension Tp at the proximal end is represented by Formula (1) below using tension Ti of each element i by the Euler's belt formula. Here, p is a friction coefficient, and θi is a bending angle of the element i.

{Formula 1}

Tₚ=Tᵢe^{μΣ}^{ₖ₌₁}^{ⁱ}θₖ (1)

The tension Ti of the element i can be calculated from Formula (2) below using the tension Tr_i at the proximal end of the wire 12 detected by the tension sensor 3. The rate of change in tension is calculated as a ratio of the tension Tr_i, which is acquired by the tension sensor 3 at the time of each displacement xi, to the initial maximum tension Tp.

$$\begin{matrix}\{ Formula 2 \} & \\ {T}_{i} = \sqrt{\frac{{T}_{p}}{{T}_{r\_i}}} & ( 2 )\end{matrix}$$

Next, in step S14, the bending angle θi of the element i is calculated. The bending angle θi is obtained from Formula (3) below.

$$\begin{matrix}\{ Formula 3 \} & \\ {θ}_{i} = \sqrt{\frac{1}{μ}} ln ( \frac{{T}_{p}}{{T}_{i}} ) - {\sum }_{k = 1}^{i - 1} {θ}_{k} & ( 3 )\end{matrix}$$

Next, in step S15, the amount of change dlk in elongation of each element k within the operation region {1, i−1} is calculated. The amount of change dlk in elongation of each element k can be calculated from the Hook's rule using a difference dTk in tension of the element k at the time of pulling and relaxing of the element k, as represented by Formula (4) below. Here, EA is rigidity of the wire 12.

dlk=Lk/EA×dTk (4)

From step S15, a total of the amount of change dl1, dl2, . . . , dl(i−1) in elongation from the element 1 to the element i−1 is obtained.

Next, in step S16, the length Li and the bending radius Ri of the element i are calculated.

Driven displacement dxi of the wire 12 is the total of the amount of change dl1, dl2, . . . , dli in elongation in an operation range {1:i} where the tension is transferred. Therefore, as represented by Formula (5) below, the amount of change dli of the element i is calculated as a difference between the displacement dxi and the total of the amount of change dl1, dl2, . . . , dli−1 in elongation from the element 1 to the element i−1.

$$\begin{matrix}\{ Formula 4 \} & \\ {dl}_{i} = {dx}_{i} - {\sum }_{k = 1}^{i - 1} {dl}_{k} & ( 5 )\end{matrix}$$

From the above, the length Li and the bending radius (curvature radius) Ri of the element i are obtained by Formulas (6) and (7) below, respectively.

$$\begin{matrix}\{ Formula 5 \} & \\ {L}_{i} = \frac{EA \cdot {dl}_{i}}{{dT}_{i}} & ( 6 )\end{matrix}$$

$${R}_{i} = \frac{{L}_{i}}{{θ}_{i}} \qquad ( 7 )$$

In the estimation of the shape of the flexible portion 13, weighting may be applied to multiple tension data. The multiple data are acquired by execution of the pulling operation and the relaxing operation for one wire 12 in multiple times, or are acquired by execution of the pulling operation and the relaxing operation for a plurality of wires 12.

Higher weighting is applied to data with high reliability among the multiple data. The data with high reliability is, for example, data acquired using the wire 12 with high linearity of spring constant, data with large tension, or data in which a driving speed of the wire 12 is slow in the relaxing operation. The greater the tension, the greater the amount of change in tension in the relaxing operation, so reliability of the data increases. Further, the slower the driving speed, the higher the data density, so reliability of the data increases.

In this way, the shape of the flexible portion 13 is estimated using the multiple data to which the weighting is applied based on the reliability, whereby errors in estimation of the shape of the flexible portion 13 can be reduced, and the shape of the flexible portion 13 can be estimated with higher accuracy.

### Second Embodiment

Next, an endoluminal device system, a control device, and a control method according to a second embodiment of the present invention will be described with reference to the drawings.

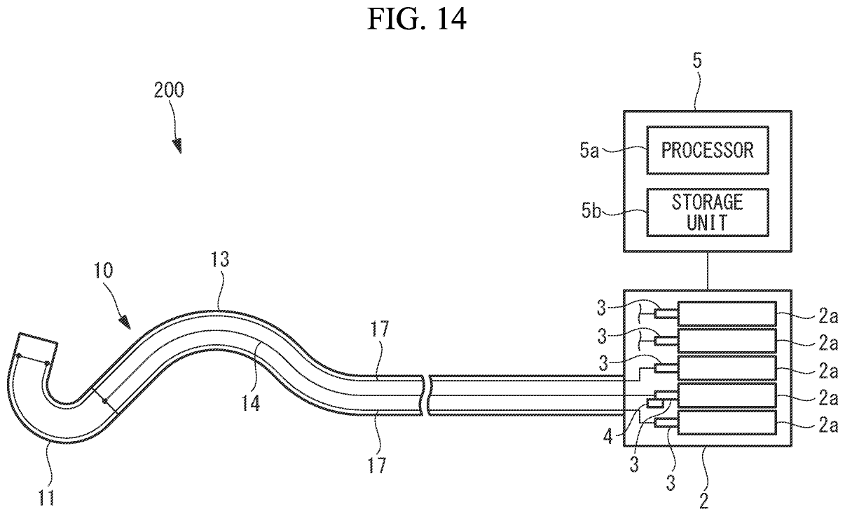

As shown in FIG. 14, an endoluminal device system 200 according to the present embodiment differs from that of the first embodiment in that a measuring wire 14 different from a driving wire 17 is provided. In the present embodiment, components different from those of the first embodiment will be described, and the components common to those of the first embodiment will be denoted by the same reference numerals, and will not be described.

The endoluminal device system 200 includes an endoluminal device 10 having a movable portion 11 and one or more driving wires (second wires) 17, a measuring wire (first wire) 14 provided in the endoluminal device 10, a driving device 2, tension sensors (first sensor) 3 that detect tension of wires 14 and 17, a displacement sensor (second sensor) 4 that detects displacement of the measuring wire 14, and a control device 5.

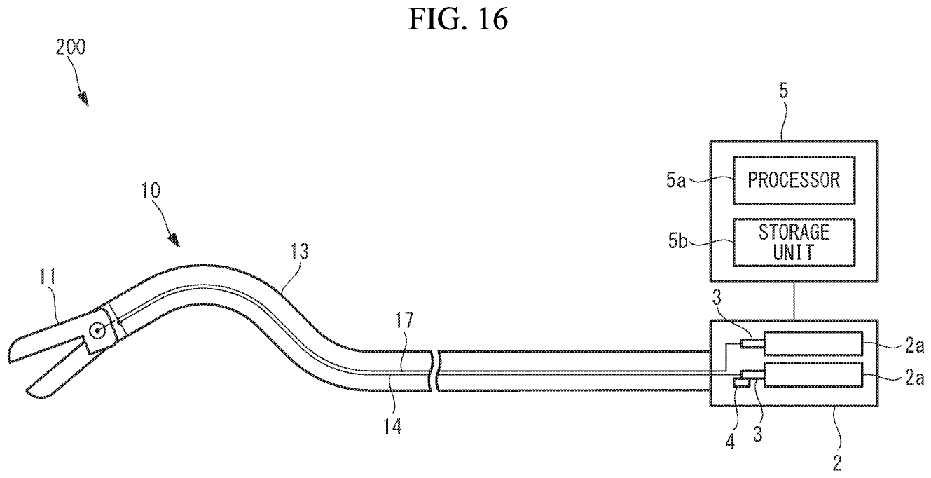

In FIG. 14, the movable portion 11 is a bending portion. As shown in FIG. 16, the movable portion 11 may be an end effector, or may be a joint.

The flexible driving wire 17 has the same configuration as the wire 12 of the first embodiment, but has a different function. In other words, the driving wire 17 of the present embodiment is disposed in the flexible portion 13 along the longitudinal direction of the flexible portion 13, a distal end of the driving wire 17 is fixed to a distal end of the bending portion 11, and a proximal end of the driving wire 17 is connected to the driving device 2. The flexible driving wire 17 extending over the entire length of the flexible portion 13 in this way bends into the same shape as the flexible portion 13 as the flexible portion 13 bends.

Unlike the wire 12 of the first embodiment, the driving wire 17 of the present embodiment has a function of transmitting the power given from the driving device 2 from the proximal end to the distal end to operate the movable portion 11, and does not have a function of acquiring shape information of the flexible portion 13. For example, four wires 17 are provided to bend the bending portion 11 upward, downward, left, and right, respectively. In the present embodiment, each of these four wires 17 is configured to be capable of functioning as a driving wire, but does not function as a measuring wire.

The flexible measuring wire 14 is disposed in the flexible portion 13 along the longitudinal direction of the flexible portion 13, a distal end of the measuring wire 14 is not connected to the movable portion 11 but is fixed to the distal end of the flexible portion 13, and a proximal end of the measuring wire 14 is connected to the driving device 2. The measuring wire 14 has a function of acquiring the shape information of the flexible portion 13, but does not have a function of transmitting the power given from the driving device 2 from the proximal end to the distal end to operate the movable portion 11.

The driving device 2 includes actuators 2a that retract and advance the proximal end portions of the wires 14 and 17 in the longitudinal direction of the wires 14 and 17, respectively.

The tension sensor 3 is provided at the proximal end portion of each of the wires 14 and 17 and detects tension at the proximal end portion of each of the wires 14 and 17.

The displacement sensor 4 detects displacement of the proximal end portion of the measuring wire 14 in the longitudinal direction of the measuring wire 14.

A friction coefficient of a path of the measuring wire 14 is greater than a friction coefficient of a path of the driving wire 17.

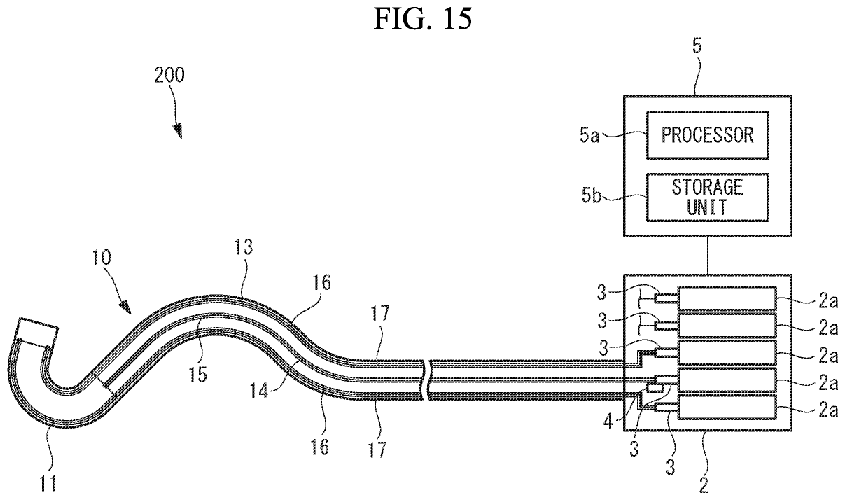

For example, as shown in FIG. 15, the endoluminal device system 200 includes a tubular first sheath 15 into which the measuring wire 14 is inserted and a tubular second sheath 16 into which the driving wire 17 is inserted. The wires 14 and 17 are movable in the longitudinal direction within the sheaths 15 and 16, respectively. A friction coefficient between the measuring wire 14 and the first sheath 15 is greater than a friction coefficient between the driving wire 17 and the second sheath 16.

Another means of making the friction coefficients of the paths of the wires 14 and 17 different from each other may include a lubricant, a stranded wire/single-strand wire, or a surface treatment.

In other words, the type of lubricant applied to the wires 14 and 17 may be selected such that the friction coefficient of the path of the measuring wire 14 is greater than the friction coefficient of the path of the driving wire 17. Alternatively, a single-strand wire may be used as the driving wire 17 and a stranded wire may be used as the measuring wire 14. Alternatively, an outer surface of the measuring wire 14 may be subjected to a surface treatment that increases the friction coefficient, for example, a surface roughening treatment that forms an uneven structure.

In the control method of the present embodiment, the processor 5a pulls and relaxes the measuring wire 14 in a pulling operation and a relaxing operation (step S2), acquires tension data and displacement data of the measuring wire 14 during the relaxing operation (step S3), and adjusts control parameters for the driving wire 17 based on the data (step S4).

As described above, according to the present embodiment, the measuring wire 14 separate from the driving wire 17 is used, and the friction acting on the measuring wire 14 is greater than the friction acting on the driving wire 17. The smaller the friction acting on the driving wire 17, the higher the transmission efficiency of power from the proximal end to the distal end of the driving wire 17. On the other hand, the greater the friction acting on the measuring wire 14, the larger the change in tension at the proximal end of the measuring wire 14 during the relaxing operation. Therefore, responsiveness of the movable portion 11 can be enhanced, sensitivity of the tension to the shape of the flexible portion 13 can be enhanced, and estimation accuracy of the shape of the flexible portion 13 and adjustment accuracy of the control parameters can be improved.

In the present embodiment, the friction coefficient of the path of the measuring wire 14 is set to be greater than the friction coefficient of the path of the driving wire 17, but alternatively or additionally, a spring constant of the measuring wire 14 may be smaller than a spring constant of the driving wire 17.

The greater the spring constant of the driving wire 17, the faster the power transmission from the proximal end to the distal end of the driving wire 17. On the other hand, the smaller the spring constant of the measuring wire 14, the larger the amount of change in displacement with respect to the change in the tension, and the larger the amount of displacement of the measuring wire 14 required in the pulling operation and the relaxing operation.

Therefore, since the spring constant of the measuring wire 14 is smaller than the spring constant of the driving wire 17, the responsiveness of the movable portion 11 can be enhanced, the sensitivity of the displacement to the shape of the flexible portion 13 can be enhanced, and the estimation accuracy of the shape of the flexible portion 13 and the adjustment accuracy of the control parameters can be improved.

In the present embodiment, the measuring wire 14 may be pulled and relaxed in a linear region of the spring characteristic of the measuring wire 14.

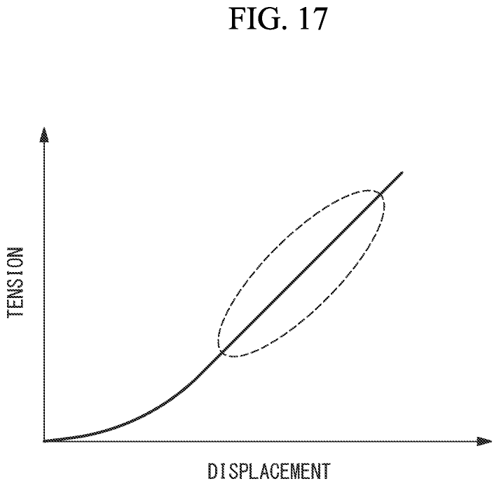

FIG. 17 shows an example of the spring characteristics of the measuring wire 14, where a horizontal axis represents the displacement of the measuring wire 14 and a vertical axis represents the tension of the measuring wire 14. When the measuring wire 14 is formed of a stranded wire, the spring characteristics may include a region where the tension varies linearly (a region enclosed by a dashed ellipse) and a region where the tension varies nonlinearly.

According to the present modified example, the linear region of the measuring wire 14 is used, and thus it is possible to simplify an arithmetic operation for the calculation of the shape of the flexible portion 13 and the adjustment of the control parameters. Further, it is possible to perform more accurately the calculation of the shape of the flexible portion 13 and the adjustment of the control parameters.

When the measuring wire 14 is formed of a single-strand wire, the measuring wire 14 has spring characteristics in which tension varies linearly over substantially the entire range of displacement. Therefore, when the single-strand wire is used as the measuring wire 14, the measuring wire 14 may be pulled and relaxed in any linear region.

### Third Embodiment

Next, an endoluminal device system, a control device, and a control method according to a third embodiment of the present invention will be described with reference to the drawings.

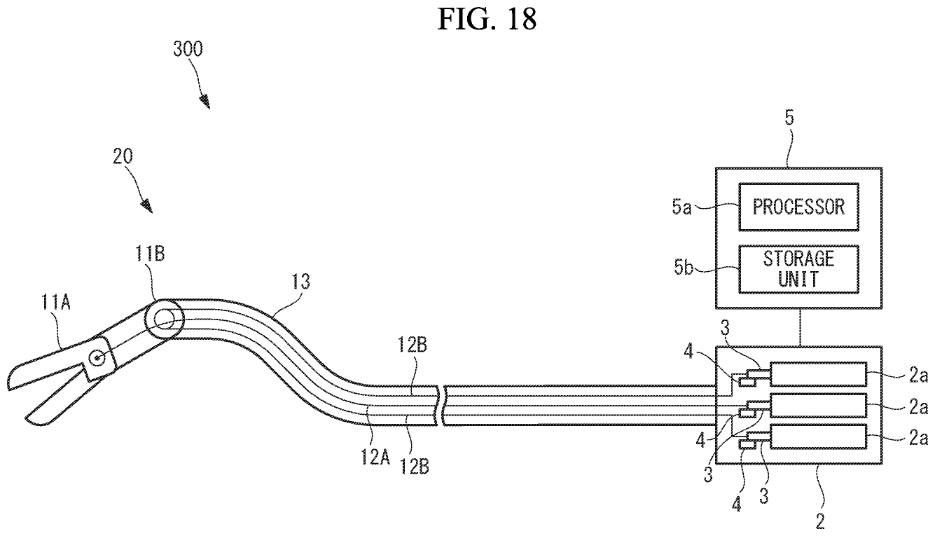

As shown in FIG. 18, an endoluminal device system 300 according to the present embodiment differs from that of the first and second embodiments in that an endoluminal device 20 includes a plurality of movable portions 11A and 11B. In the present embodiment, components different from those of the first and second embodiments will be described, and the components common to those of the first and second embodiments will be denoted by the same reference numerals, and will not be described.

The endoluminal device system 300 includes an endoluminal device 20 having two movable portions 11A and 11B and two or more wires (first wires) 12A and 12B, a driving device 2 that drives movable portions 11A and 11B by pulling and relaxing the wires 12A and 12B, a tension sensor (first sensor) 3 that detects tension of each of the wires 12A and 12B, a displacement sensor (second sensor) 4 that detects displacement of each of the wires 12A and 12B, and a control device 5.

The movable portions 11A and 11B are, for example, any one of bending portions, end effectors, and joints. In the example of FIG. 18, the movable portion 11A is an end effector, the movable portion 11B is a joint arranged between the end effector 11A and a distal end of a flexible portion 13.

Similarly to the wires 12 of the first embodiment, the wires 12A and 12B are driving wires for operating the corresponding movable portions 11A and 11B, and are also measuring wires. Distal ends of the wires 12A and 12B are fixed to the corresponding movable portions 11A and 11B, respectively, and a proximal end of each of the wires 12A and 12B is connected to the driving device 2.

The driving device 2 includes two or more actuators 2a that retract and advance the proximal end portions of the wires 12A and 12B in the longitudinal direction of the wires 12A and 12B, respectively.

The tension sensor 3 is provided at the proximal end portion of each of the wires 12A and 12B and detects tension at the proximal end portion of each of the wires 12A and 12B.

The displacement sensor 4 detects displacement of the proximal end portion of each of the wires 12A and 12B in the longitudinal direction of each of the wires 12A and 12B.

In the present embodiment, the processor 5a executes a control method to adjust control parameters of the movable portions 11A and 11B when operating either one of the movable portions 11A and 11B. For example, the processor 5a acquires tension data and displacement data of the wire 12B during the relaxing operation of the wire 12B accompanying the operation of the movable portion 11B, and adjusts control parameters for both the movable portions 11A and 11B based on the acquired data.

As described above, according to the present embodiment, when one of the movable portions 11A and 11B performs an operation necessary for work such as treatment, control parameters for the other of the movable portions 11A and 11B can also be adjusted.

In the present embodiment, the processor 5a may execute the control method in response to a trigger or may repeatedly execute the control method at predetermined time interval, as in the first embodiment. In this case, the processor 5a may select any pair of wires from a predetermined plurality of wires 12A and 12B, and may pull and relax the selected wire in the pulling operation and the relaxing operation.

In the present embodiment, the processor 5a may calculate the shape of the flexible portion 13 and adjust the control parameters based on multiple tension data. The multiple data are acquired by execution of the pulling operation and the relaxing operation for one measuring wire 12 in multiple times, or are acquired by execution of the pulling operation and the relaxing operation for a plurality of measuring wires 12. With such a configuration, it is possible to improve the accuracy in calculation of the shape of the flexible portion 13 and adjustment of the control parameter.

Also in the first and second embodiments described above, the processor 5a may calculate the shape of the flexible portion 13 and adjust the control parameters based on the multiple tension data.

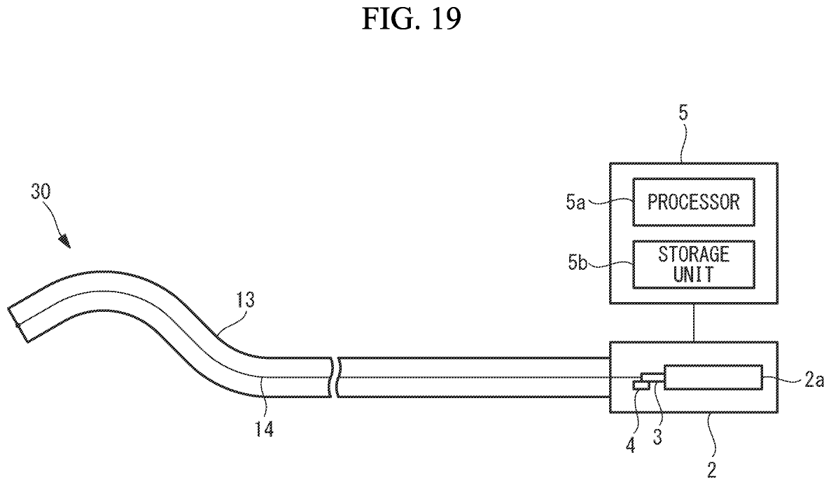

In the first to third embodiments described above, the endoluminal devices 1, 10, and 20 include the movable portions 11, 11A, and 11B, respectively, but the endoluminal device 30 may not include the movable portion as shown in FIG. 19. The distal end of the measuring wire 14 is fixed to the distal end of the flexible portion 13. In this case, after acquiring the tension data and the displacement data of the measuring wire 14, the processor 5a may calculate the shape of the flexible portion 13 and may not perform the adjustment of the control parameters.

### Fourth Embodiment

Next, an endoluminal device system, a control device, and a control method according to a fourth embodiment of the present invention will be described with reference to the drawings.

An endoluminal device system according to the present embodiment differs from those of the first to third embodiments in that the control device 5 causes the driving device 2 to automatically execute a relaxing operation without movement of the movable portion 11. In the present embodiment, components different from those of the first to third embodiments will be described, and the components common to those of the first to third embodiments will be denoted by the same reference numerals, and will not be described.

A control method of the endoluminal device according to the present embodiment can be applied to both the endoluminal device system 100 of the first embodiment including the wire 12 and the endoluminal device 300 including the wires 12A and 12B. In the present embodiment, the endoluminal device system 100 of the first embodiment will be described as an example.

As described above, in the endoluminal device 1 such as the electric endoscope having the elongated flexible portion 13, since dynamic characteristics of the wire 12 change due to the bending of the flexible portion 13, it is necessary to adjust the control parameters according to the shape of the flexible portion 13 in order to realize electric control. As one means for solving the problem, a method has been proposed in which an operator selects from a plurality of pre-registered shapes of the flexible portion or a plurality of treatment sites and a processor 5a changes control parameters according to the selected shape or treatment site (for example, see US Unexamined Patent Application, Publication No. 2017/0340400). In such a method, it is necessary for the operator to perform the manipulation of selecting the shape or treatment site.

As will be described in detail below, in the control method of the present embodiment, it is not necessary for the operator to perform an operation of inputting the trigger in step S1, and the control parameters are automatically adjusted.

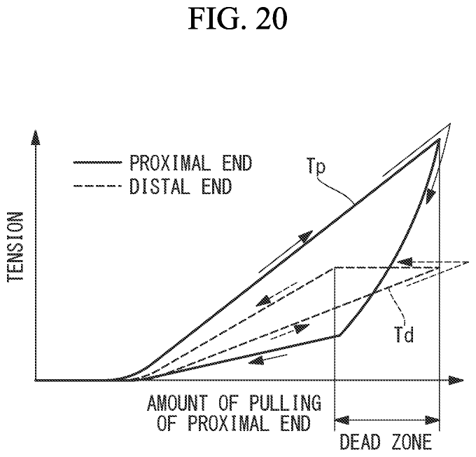

FIG. 20 shows a relationship between the amount of pulling (displacement) of the proximal end of the wire 12 and tensions Tp and Td at the proximal and distal ends of the wire 12. In FIG. 20, arrows indicate directions of change in the amount of pulling. As can be seen from FIG. 20, the change in tension during pulling is different from the change in tension during relaxing, and accordingly, the tension Tp and Td changing according to the pulling and the relaxing of the proximal end draw a hysteresis loop. Early in relaxing, a dead zone is generated due to friction in which the decrease in tension Tp at the proximal end is not transferred to the distal end. The dead zone can be determined according to the shape of the flexible portion 13, and can be calculated from the shape of the flexible portion 13.

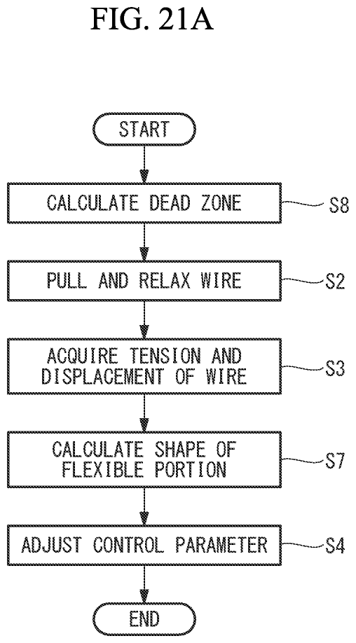

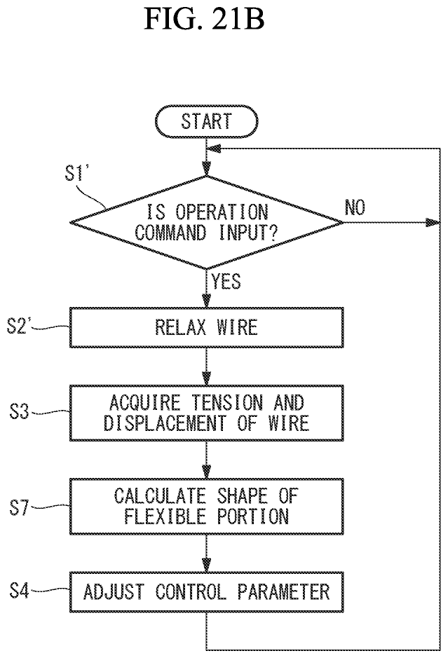

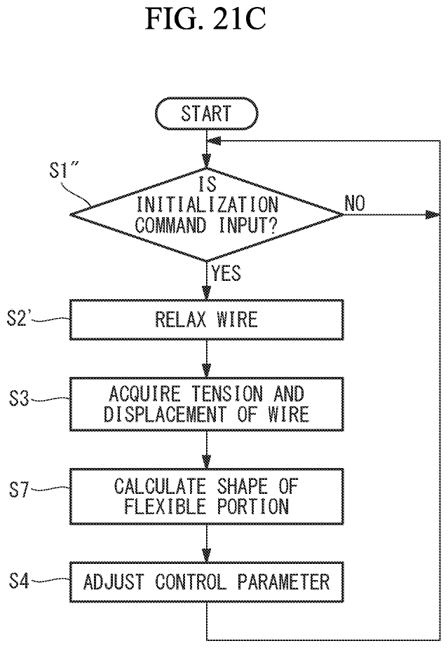

The processor 5a executes a control method shown in FIG. 21A at arbitrary timing, for example, at predetermined time intervals or according to a predetermined schedule set by the operator.

Specifically, the processor 5a acquires the last estimated shape of the flexible portion 13 from the storage unit 5b, and calculates the dead zone from the shape of the flexible portion 13 (step S8).

Next, the processor 5a controls the driving device 2 to retract and advance the proximal end of the wire 12 within the dead zone, thereby executing the pulling and relaxing operations of the wire 12 (step S2). Preferably, the processor 5a advances the proximal end of the wire 12 to a position where the dead zone ends.

Next, the processor 5a acquires tension and displacement of the wire 12 during the relaxing operation (step S3), estimates the shape of the flexible portion 13 (step S7), and causes the storage unit 5b to store the estimated shape.

As described above, as long as the proximal end of the wire 12 moves within the dead zone, since the decrease in tension is not transmitted to the distal end, the pulling operation and the relaxing operation of the wire 12 are executed in step S2 without movement of the movable portion 11 such as the bending portion. In other words, the execution of step S2 does not affect the manipulation of the movable portion 11 by the operator. Therefore, the processor 5a can automatically execute steps S2, S3, S7, and S4 at arbitrary timings without requiring the manipulation by the operator and without being noticed by the operator.

FIGS. 21B and 21C show other control methods for automatically executing a relaxing operation without movement of the movable portion 11.

In the control method of FIG. 21B, an operation of the movable portion 11 in a direction in which any wire 12 relaxes is used as the relaxing operation. For example, when the bending angle or the bending direction of the bending portion 11 is changed, any wire 12 relaxes. When an operation command for the movable portion 11 in the direction in which any wire 12 relaxes is input to the manipulation device 6 (step S1′), the processor 5a controls the driving device 2 to operate the movable portion 11 according to the operation command, and executes the relaxing operation while the movable portion 11 is being operating (step S2′). The processor 5a acquires tension data and displacement data of the wire 12 that relaxes during the operation of the movable portion 11 (step S3), and executes steps S4 and S7 using the acquired data.

According to the control method of FIG. 21B, the tension data and displacement data are acquired during the relaxing operation that is executed simultaneously with the operation of the movable portion 11 by the operator. Therefore, it is possible to automatically execute acquisition of the data and adjustment of the control parameters without the input of the trigger to the control device 5 from the operator.

In the control method of FIG. 21C, an initialization operation for returning the movable portion 11 to the initial state is used as the relaxing operation.

As one method of initializing the bending portion 11, there is a method of controlling the bending of the bending portion 11 based on a difference in tension between a pair of wires 12 for bending in opposite directions. When the difference in tension is equal to or greater than a threshold, the processor 5a changes the bending angle of the bending portion 11 in a direction in which the difference in tension is smaller. Therefore, one of the pair of wires 12 relaxes in the initialization operation. When the difference in tension is less than the threshold, the bending portion 11 is restored to a straight shape.

When the operator inputs an initialization command to the control device 5 (step S1″), the processor 5a controls the driving device 2 to return the movable portion 11 to the initial state according to the initialization command, and executes relaxing operation during the initialization operation (step S2′). The processor 5a acquires tension data and displacement data of the wire 12 that relaxes during the initialization operation of the movable portion 11 (step S3), and executes steps S4 and S7 using the acquired data.

According to the control method of FIG. 21C, the tension data and the displacement data are acquired during the relaxing operation that is executed simultaneously with the initialization operation of the movable portion 11. Therefore, it is possible to automatically execute acquisition of the data and adjustment of the control parameters without the input of the trigger to the control device 5 from the operator.

### Fifth Embodiment

Next, an endoluminal device system, a control device, and a control method according to a fifth embodiment of the present invention will be described with reference to the drawings.

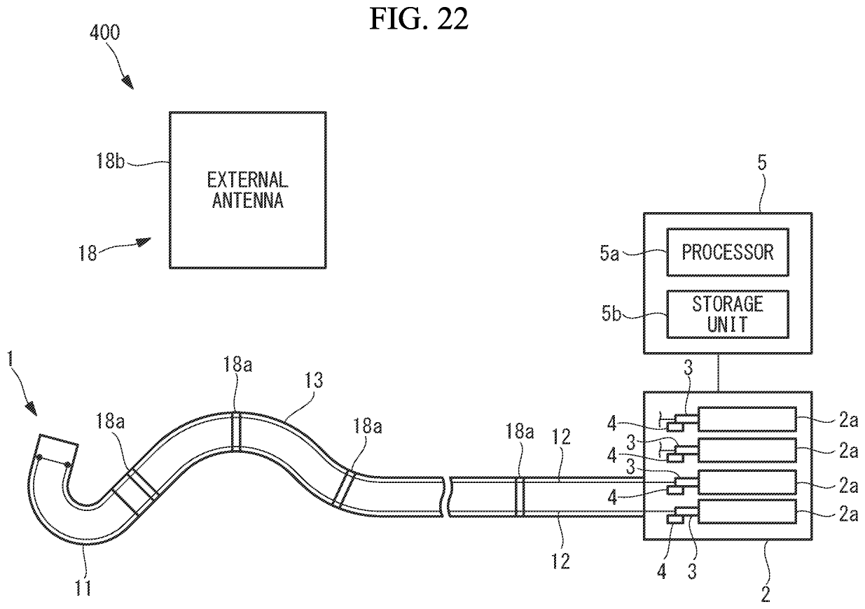

As shown in FIG. 22, an endoluminal device system 400 according to the present embodiment differs from those of the first to fourth embodiments in that a shape detection device 18 is further provided to detect a shape of an endoluminal device. In the present embodiment, components different from those of the first to fourth embodiments will be described, and the components common to those of the first to fourth embodiments will be denoted by the same reference numerals, and will not be described.

The endoluminal device system 400 includes a shape detection device 18 in addition to the endoluminal device 1, the wire 12, the driving device 2, the sensors 3 and 4, and the control device 5.

The system 400 shown in FIG. 22 is based on the system 100 of the first embodiment, but may be based on the system 200 of the second embodiment or the system 300 of the third embodiment. In other words, the system 400 may include the endoluminal device 10, 20, or 30 instead of the endoluminal device 1.

The shape detection device 18 includes a plurality of internal sensors 18a mounted on the endoluminal device 1 and an external antenna 18b arranged outside the endoluminal device 1.

The plurality of internal sensors 18a are magnetic coils that generate magnetism, and are arranged in the longitudinal direction of the flexible portion 13.

The external antenna 18b is, for example, a UPD (Endoscope Position Detecting Unit). The external antenna 18b has a reception range (detection range) that is a three-dimensional space having a predetermined size, and the internal sensor 18a from which the external antenna 18b can receive magnetism is only the internal sensor 18a within the reception range. The external antenna 18b receives the magnetism generated by each of the internal sensors 18a within the reception range, calculates a position of each of the internal sensors 18a based on the magnetism, and calculates the shape of the flexible portion 13 by connecting the positions of the plurality of internal sensors 18a with lines.

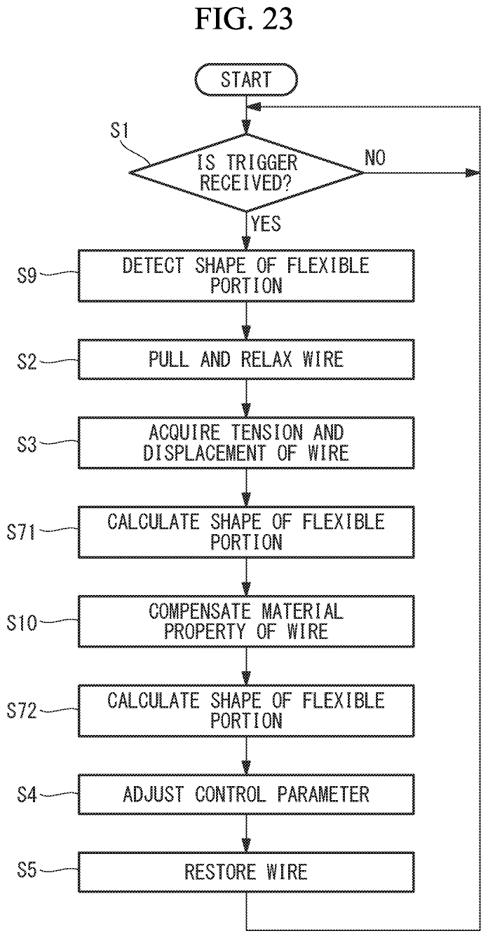

As shown in FIG. 23, a control method of the present embodiment includes steps S1, S2, S3, S4, and S5, step S9, and step S71.

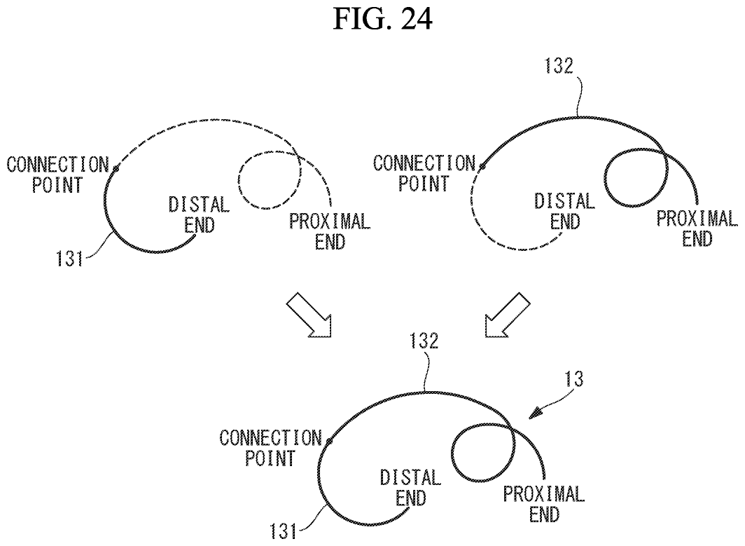

After step S1, the processor 5a causes the shape detection device 18 to detect a shape of a distal side portion 131 of the flexible portion 13, and acquires the shape of the distal side portion 131 from the shape detection device 18 (step S9). As shown in FIG. 24, the distal side portion 131 is a portion from the distal end to a connection point that is disposed within the reception range of the shape detection device 18.

After steps S2 and S3, the processor 5a estimates a shape of at least a proximal side portion 132 of the flexible portion 13 of the endoluminal device 1 using the tension data and the displacement data, and estimates a whole shape of the flexible portion 13 from the shape of the distal side portion 131 and the shape of the proximal side portion 132 (step S71).

For example, as shown in FIG. 24, the processor 5a may estimate the shape of only the proximal side portion 132 using the tension data and the displacement data, and may interconnect the distal side portion 131 detected by the shape detection device 18 and the estimated proximal side portion 132 at a connection point. Alternatively, the processor 5a may estimate a whole shape of the endoluminal device 13 using the tension data and the displacement data, and may replace the estimated distal side portion 131 with the distal side portion 131 detected by the shape detection device 18.

Depending on the amount of insertion of the flexible portion 13 into the body, a length of the distal side portion 131 disposed within the reception range changes, and accordingly the number of internal sensors 18a disposed within the reception range changes. The processor 5a may calculate the length of each of the distal side portion 131 and the proximal side portion 132 from the number of internal sensors 18a from which the magnetism is received by the external antenna 18b. Alternatively, the length of each of the distal side portion 131 and the proximal side portion 132 may be determined in advance based on the dimensions of the reception range.

The use of the shape detection device 18 is advantageous in that the shape of the flexible portion 13 can be detected with high accuracy, but is disadvantageous in that a spatial range capable of being measured with high accuracy is limited to the reception range of the external antenna 18b. Therefore, it is particularly difficult to detect the shape of the entire length of the electrically driven long flexible portion 13 with high accuracy.

The method of estimating the shape of the flexible portion 13 from the tension data and the displacement data of the wire 12 during the relaxing operation is advantageous in that the shape can be estimated over the entire length of the flexible portion 13 regardless of the length of the flexible portion 13, but is disadvantageous in that the estimation accuracy of the shape of the distal side of the flexible portion 13 is low. This is because the shape of the flexible portion 13 is estimated in order from the proximal end to the distal end, and thus the estimation error on the distal side of the flexible portion 13 increases. Furthermore, estimation errors due to individual differences in material properties of the wire 12 also occur.

According to the present embodiment, the shape of the distal side portion 131 is detected with high accuracy by the shape detection device 18, and the shape of the proximal side portion 132 is estimated with high accuracy using the tension data and the displacement data. By a combination of the shapes of the two portions 131 and 132 acquired by different methods, the whole shape of the flexible portion 13 can be estimated with high accuracy.

In the present embodiment, the processor 5a may compensate the material properties of the wire 12, for example, rigidity and friction coefficient, using the shape of the distal side portion 131 acquired by the shape detection device 18 (step S10). There are individual differences in the material properties of the wire 12. By compensation of the material properties, it is possible to improve the estimation accuracy of the shape of the flexible portion 13 using the tension data and the displacement data.

Specifically, in step S10, the processor 5a selects a highly accurate portion, which is a portion detected in particular with high accuracy, from the shapes of the flexible portion 13 detected by the shape detection device 18. For example, the processor 5a specifies and selects a highly accurate portion based on a distance from the external antenna 18b to each internal sensor 18a or a variation in the calculated position of each internal sensor 18a. Next, the processor 5a selects a portion corresponding to the highly accurate portion from the shapes of the flexible portion 13 estimated using the tension data and the displacement data. Therefore, in step S71, the shape of the entire length of the flexible portion 13 is estimated. The processor 5a compensates the material properties such that the shape of the corresponding portion matches the shape of the highly accurate portion.

After compensating the material properties, the processor 5a may estimate the shape of the proximal side portion 132 again from the tension data and the displacement data using the compensated material properties (step S72). Thus, it is possible to obtain the shape that is estimated with higher accuracy than the shape estimated in step S71.

### Sixth Embodiment

Next, an endoluminal device system, a control device, and a control method according to a sixth embodiment of the present invention will be described with reference to the drawings.

An endoluminal device system according to the present embodiment is characterized by a calculation method of control parameters in step S5 executed by the control device 5. The calculation method of control parameters of the present embodiment can be implemented in combination with any of the first to fifth embodiments. In the present embodiment, components different from those of the first to fifth embodiments will be described, and the components common to those of the first to fifth embodiments will be denoted by the same reference numerals, and will not be described.

Conventionally, a method has been proposed for accurately estimating control parameters of a complicatedly bent wire. For example, in Japanese Unexamined Patent Application, Publication No. 2015-154814, a flexible portion is divided into a plurality of sections, dynamic characteristics are estimated for each section, and control parameters are calculated using the estimated dynamic characteristics. In this case, the greater the number of sections, the higher the accuracy of the control parameters, but enormous calculation is required.

The method of the present embodiment is to estimate the dynamic characteristics of the complicatedly bent wire and to calculate the control parameters with a small amount of calculation.

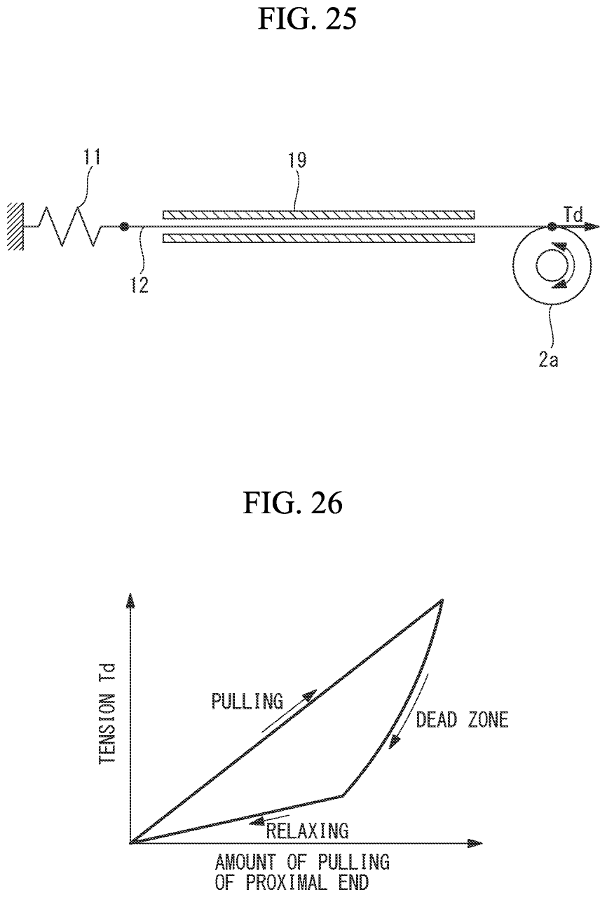

FIG. 25 shows a model of an endoluminal device used in the present embodiment. A movable portion 11 such as a bending portion is defined as a spring. A wire 12 passes through an elongated sheath 19, and a proximal end of the wire 12 is displaced by an actuator 2a such as a motor. The wire 12 is stretchable in the longitudinal direction, and friction exists between the wire 12 and the sheath 19.

As shown in FIG. 26, the state of the wire 12 is classified into three states of pulling, relaxing, and dead zone. In the pulling and relaxing states, tension Td at the proximal end of the wire 12 changes linearly or substantially linearly with respect to the displacement of the proximal end, and thus the dynamic characteristics such as a friction coefficient of the wire 12 are constant or substantially constant. On the other hand, in the dead zone state, the tension Td changes nonlinearly with respect to the displacement of the proximal end, and the dynamic characteristics of the wire 12 change. The dynamic characteristics correspond to a slope of a graph in FIG. 26.

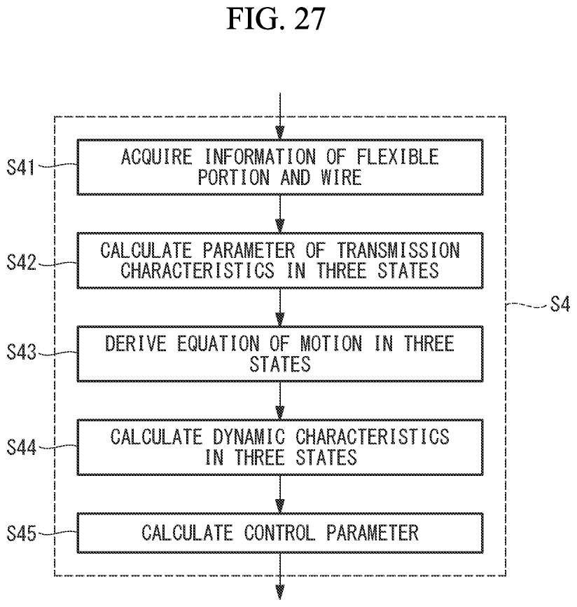

FIG. 27 shows step S4 in the control method of the present embodiment.

The processor 5a acquires a shape of the flexible portion 13 and displacement of the wire 12 (step S41). The processor 5a may also further acquire tension of the wire 12.

Next, the processor 5a calculates parameters of power transmission characteristics of the wire 12 in each of the three states, based on the shape and the displacement (step S42). Specifically, the processor 5a calculates, from the shape of the flexible portion 13, transmission efficiency of the tension of the wire 12 in each of the three states, and calculates a spring constant of the wire 12 in each of the three states from the displacement and transmission efficiency of the wire 12.

Next, the processor 5a derives a relationship between the amount of pulling and the tension in each of the pulling and relaxing states, using the spring constant of the wire 12, and derives a relationship between the amount of pulling and the tension in the dead zone state, as a difference between the relationships in the two states of pulling and relaxing (step S43). In other words, in step S43, the processor 5a obtains an equation of motion Mu″=T in each of the three states. Here, M indicates the mass of the wire 12, u indicates the displacement of the proximal end of the wire 12, and T indicates the tension of the wire 12.

Next, the processor 5a calculates the dynamic characteristics of the wire 12 in the pulling state from the equation of motion in the pulling state, and calculates the dynamic characteristics of the wire 12 in the relaxing state from the equation of motion in the relaxing state (step S44). Further, the processor 5a calculates a range of the displacement amount of the wire 12 corresponding to the dead zone (step S44).

Next, the processor 5a calculates control parameters, for example, for operating the bending portion 11, using the dynamic characteristics in the pulling or relaxing state (step S45). The processor 5a may relax the wire 12 by the amount corresponding to the dead zone after operating the bending portion 11 by pulling the wire 12. Thus, since there is no dead zone at the time of next operation of the bending portion 11, responsiveness of the bending portion 11 can be improved.

As described above, according to the present embodiment, the state of the wire 12 is classified into the three states, and three equations of motion are used according to the states of the wire 12. Thus, the dynamic characteristics can be calculated with the entire wire 12 as one element, and the dynamic characteristics can be estimated and the control parameters can be calculated with a small amount of calculation.

### Seventh Embodiment

Next, an endoluminal device system, a control device, and a control method according to a seventh embodiment of the present invention will be described with reference to the drawings.

An endoluminal device system of the present embodiment is characterized by a calculation method of control parameters in step S5 executed by the control device 5. The calculation method of control parameters of the present embodiment can be implemented in combination with any of the first to sixth embodiments. In the present embodiment, components different from those of the first to sixth embodiments will be described, and the components common to those of the first to sixth embodiments will be denoted by the same reference numerals, and will not be described.

Mounting a sensor on a distal end portion of an endoluminal device such as an electric endoscope has design limitations. Therefore, a spring model is used as a control model instead of the sensor to estimate a state S of the wire 12, for example, tension Td at a distal end.

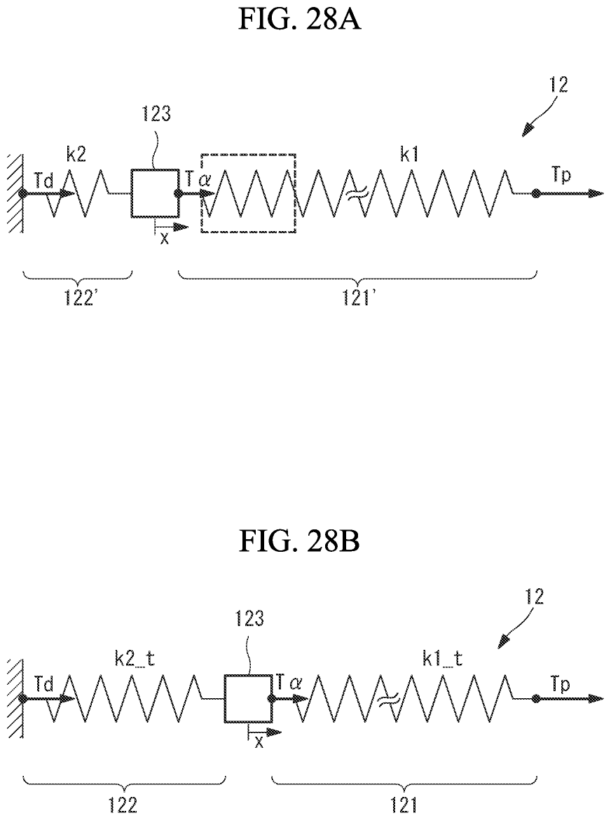

FIG. 28A shows a general spring model. In the spring model, the wire 12 stretchable in the longitudinal direction is defined by two springs 121′ and 122′ and a mass point 123 connecting the two springs 121′ and 122′. Specifically, a part of the wire 12 disposed within the flexible portion 13 is defined as a long first spring 121′ having a first spring constant k1. The other part of the wire 12 disposed within the movable portion 11 such as a bending portion is defined as a short second spring 122′ having a second spring constant k2. A distal end of the first spring 121′ is connected to a proximal end of the second spring 122′ by the mass point 123. Tp indicates tension at the proximal end of the wire 12, and Td indicates tension at the distal end of the wire 12.

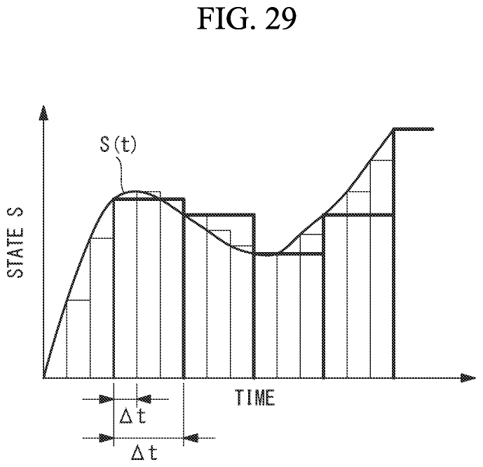

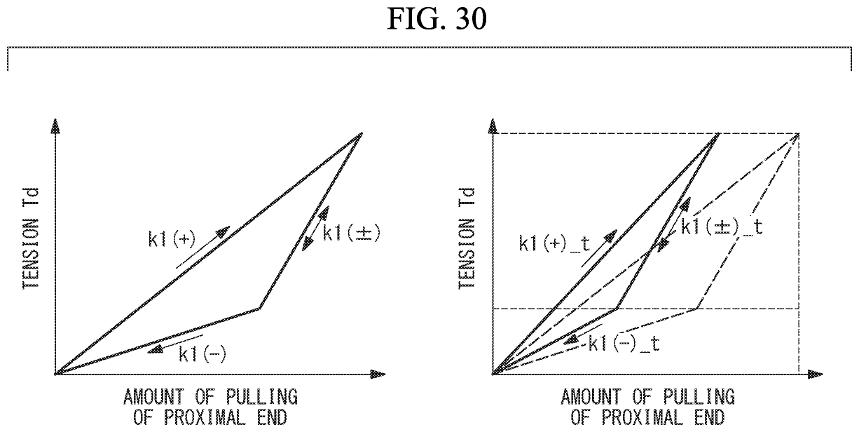

As shown in FIG. 29, in step S4, the processor 5a calculates a state S(t) of the wire 12, for example, tension at time t every step time Δt. In order to control the endoluminal device 1 in real time, it is necessary to calculate the state S of the wire 12 and control parameters in real time. For this reason, one means is to reduce the number of times of calculation by lengthening the step time Δt, thereby shortening the total calculation time.

When the step time Δt is lengthened for high-speed calculation of the spring model, displacement Δx of the mass point 123 at the step time Δt becomes large. Here, since the second spring 122′ is much shorter than the first spring 121′, the second spring constant k2 is greater than the first spring constant k1. Therefore, the tension Td at the distal end changes in response to movement of the mass point 123 with very high sensitivity. For this reason, when the step time Δt is lengthened, the tension Td abruptly changes and oscillates. In other words, the general spring model is unstable with respect to the long step time Δt, and calculation accuracy of the tension Td becomes unstable as the calculation speed increases.

As a means for solving the instability of calculation, a method has been proposed in which the stability of calculation with respect to the step time Δt is determined and the step time Δt is adjusted according to the determination result. For example, in the Publication of Japanese Patent No. 5692739, calculation is executed with a step time of “long”, the stability of the calculation is determined every time, and the step time is shortened when it is determined to be unstable.

Such a method is not to stabilize the model itself. Therefore, the total calculation time can be shortened in a case of a stable model, but the total calculation time is lengthened in a case of an unstable model. For this reason, such a method is effective in that the total calculation time can be shortened in off-line simulation, but is difficult to be used in real-time simulation.

In the present embodiment, an improved spring model shown in FIG. 28B is used as a control model instead of the general spring model. In the improved spring model, a position of a mass point 123 is changed proximally, and thus a part (a dashed rectangular part in FIG. 28A) of a first spring 122 is virtually shifted to a second spring 122. Thus, the second spring 122 becomes virtually longer, and a second spring constant k2_t becomes virtually smaller than a spring constant k2.

Specifically, the second spring constant k2_t in the improved spring model is represented by the following formula. Here, k2 indicates a true second spring constant (an actual spring constant of a part of the wire 12 disposed on the movable portion 11), and kt indicates a spring constant of the part shifted from the first spring 121 to the second spring 122.

k2_t=k2×kt/(k2+kt)

In the improved spring model, the sensitivity of the tension Td to the amount of elongation (displacement x) of the wire 12 is reduced by kt/(k2+kt) times (<1.0) compared to the general spring model.

In the improved spring model, as described above, a part of the first spring 121 is moved to the second spring 122 by movement of the mass point 123, and thus the second spring constant k2_t becomes smaller. Thereby, the improved spring model is obtained that is stable compared to the general spring model. By using such an improved spring model, oscillation of the tension Td can be prevented even when the step time Δt is lengthened, and the calculation accuracy of the tension Td in real time can be stabilized.

As a part of the first spring 121 is shifted to the second spring 122, the first spring constant k1 also needs to be adjusted to maintain the relationship between the tension Tp at the proximal end and the tension Td at the distal end. Therefore, the adjusted first spring constant k1_t is used in the improved spring model. Thus, in the improved spring model, the relationship between the tension Tp and the tension Td as in the general spring model can be maintained.

The friction acting on the wire 12 during the pulling of the wire 12 is different from that during the relaxing of the wire 12. Therefore, in the spring model, the relationship between the displacement x and the tension Td during the pulling when tension Ta is acting on the mass point 123 differs from that during the relaxing.

As shown in FIG. 30, the relationship between the displacement x and the tension Td can be represented using three spring constants k1(+), k1(±), and k1(−) of the first spring 121′ according to the amount of pulling (displacement x) of the proximal end. A left diagram of FIG. 30 shows a relationship between the displacement x and the tension Td in the general spring model. Here, k1(+), k1(±), and k1(−) indicate the first spring constants during pulling, dead zone, and relaxing of the wire 12, respectively.

As shown in a right diagram of FIG. 30, three spring constants k1(+)_t, k1(±)_t, and k1(−)_t of the first spring 121 are also used in the improved spring model. The first spring constants k1(+)_t, k1(±)_t, and k1(−)_t during relaxing, dead zone, and relaxing are as follows.

$$\begin{matrix}\{ Formula 6 \} & \\ k 1 ( + ) \_t = \frac{k 1 * k t}{k t - α * k 1 ( + )} & \end{matrix}$$

$$k 1 ( \pm ) \_t = {( \frac{1}{α * k 1 ( + )} \frac{α}{α * k 1 ( - )} )}^{- 1}$$

$$k 1 ( - ) \_t = \frac{α * k 1 ( - ) * k t}{α * k t - k 1 ( - )}$$

The tension is lost due to the friction while being transferred between both ends of the wire 12. Here, a indicates transmission efficiency (a ratio of the tension at the distal end to the tension at the proximal end) of the tension from the proximal end to the distal end of the first spring 121. The first spring constants k1(+)_t, k1(±)_t, and k1(−)_t are calculated in consideration of the transmission efficiency α.

In this way, by using the three spring constants k1(+)_t, k1(±)_t, and k1(−)_t in consideration of the transmission efficiency α, the tension Td in each state of the wire 12 can be calculated in consideration of the friction.

The embodiments of the present invention and the modified examples thereof have been described above in detail with reference to the drawings, but the detailed configuration is not limited to the above-described embodiments and includes design changes made without departing from the scope of the present invention. Further, the components of the above-described embodiments and modified examples can be combined as appropriate.

### REFERENCE SIGNS LIST

1, 10, 20 endoluminal device
2 driving device
3 tension sensor (first sensor)
4 displacement sensor (second sensor)
5 control device
5a processor
11 bending portion, movable portion
11A, 11B movable portion
12 wire (first wire)
12A, 12B (first wire)
13 flexible portion
14 measuring wire (first wire)
15 first sheath
16 second sheath
17 driving wire (second wire)
18 shape detection device
100, 200, 300, 400 endoluminal device system

1. An endoluminal device system comprising:
an elongated and flexible endoluminal device;
a flexible first wire that is disposed in the endoluminal device along a longitudinal direction of the endoluminal device;
a first sensor that detects tension of the first wire;
a second sensor that detects displacement of the first wire;
a driving device capable of pulling the first wire; and
at least one processor, wherein
the at least one processor is configured to:
cause the driving device to execute a relaxing operation for relaxing the first wire,
acquire the tension of the first wire during the relaxing operation detected by the first sensor,
acquire the displacement of the first wire during the relaxing operation detected by the second sensor, and
adjust a control parameter of the endoluminal device based on a rate of change in the tension with respect to the displacement during the relaxing operation.

2. The endoluminal device system according to claim 1, wherein the endoluminal device includes a flexible portion that is elongated and flexible and a movable portion connected to a distal end of the flexible portion,
the first wire is coupled to the movable portion, and
the movable portion moves by pulling and relaxing of the first wire.

3. The endoluminal device system according to claim 2, wherein the at least one processor is configured to cause the driving device to execute the relaxing operation without movement of the movable portion.

4. The endoluminal device system according to claim 3, wherein the at least one processor is configured to control the driving device to displace a proximal end of the first wire within a dead zone, thereby causing the driving device to execute the relaxing operation without the movement of the movable portion.

5. The endoluminal device system according to claim 3, wherein the at least one processor is configured to cause the driving device to execute the relaxing operation during an operation of the movable portion in which the first wire is relaxed.

6. The endoluminal device system according to claim 3, wherein the at least one processor is configured to causes the driving device to execute the relaxing operation during an initialization operation of the movable portion in which the first wire is relaxed.

7. The endoluminal device system according to claim 3, wherein the endoluminal device system includes a plurality of the first wires,
the movable portion is a bending portion, the plurality of first wires being coupled to the bending portion, and
the at least one processor is configured to:
recognize a first wire being driven that is being pulled for bending of the bending portion among the plurality of first wires before executing the relaxing operation, and
cause the driving device to execute the relaxing operation without the movement of the movable portion by simultaneously relaxing the first wire being driven and the first wire opposing the first wire being driven.

8. The endoluminal device system according to claim 3, wherein the endoluminal device system includes a plurality of the first wires,
the movable portion is a bending portion, the plurality of first wires being coupled to the bending portion, and
the at least one processor is configured to:
recognize a first wire being driven that is being pulled for bending of the bending portion among the plurality of first wires before executing the relaxing operation, and
cause the driving device to execute the relaxing operation without the movement of the movable portion by relaxing any of the first wires other than the first wire being driven.

9. The endoluminal device system according to claim 1, wherein the endoluminal device includes a flexible portion that is elongated and flexible, a movable portion connected to a distal end of the flexible portion, and a second wire that is disposed in the flexible portion along a longitudinal direction of the flexible portion,
the first wire is not coupled to the movable portion, and
the second wire is coupled to the movable portion.

10. The endoluminal device system according to claim 9, further comprising:
a first sheath into which the first wire is inserted; and
a second sheath into which the second wire is inserted, wherein
a friction coefficient between the first wire and the first sheath is greater than a friction coefficient between the second wire and the second sheath.

11. The endoluminal device system according to claim 9, wherein a spring constant of the first wire is smaller than a spring constant of the second wire.

12. The endoluminal device system according to claim 1, wherein the control parameter is the amount of pulling of the first wire and the amount of relaxing of the first wire for transferring tension to a distal end of the first wire.

13. The endoluminal device system according to claim 1, wherein the at least one processor is configured to estimate a shape of the endoluminal device based on the rate of change.

14. The endoluminal device system according to claim 13, further comprising a shape detection device that detects a shape of a distal side portion of the endoluminal device disposed within a detection range, wherein
the at least one processor is configured to estimate a whole shape of the endoluminal device from the shape of the distal side portion detected by the shape detection device and a shape of a proximal side portion of the endoluminal device estimated based on the rate of change.

15. A control device for an endoluminal device system, the endoluminal device system including: an elongated and flexible endoluminal device; a flexible first wire that is disposed in the endoluminal device along a longitudinal direction of the endoluminal device; a first sensor that detects tension of the first wire; a second sensor that detects displacement of the first wire; and a driving device capable of pulling the first wire, wherein
the control device comprises at least one processor, and wherein
the at least one processor is configured to:
cause the driving device to execute a relaxing operation for relaxing the first wire,
acquire the tension of the first wire during the relaxing operation detected by the first sensor,
acquire the displacement of the first wire during the relaxing operation detected by the second sensor, and
adjust a control parameter of the endoluminal device based on a rate of change in the tension with respect to the displacement during the relaxing operation.

16. The control device according to claim 15, wherein the endoluminal device includes a movable portion, and wherein
the at least one processor is configured to cause the driving device to execute the relaxing operation without movement of the movable portion.

17. The control device according to claim 16, wherein the endoluminal device system includes a plurality of the first wires, the movable portion is a bending portion, and the plurality of first wires are coupled to the bending portion, and wherein
the at least one processor is configured to:
recognize a first wire being driven that is being pulled for bending of the bending portion among the plurality of first wires before executing the relaxing operation, and
simultaneously relax the first wire being driven and the first wire opposing the first wire being driven in the relaxing operation.

18. A control method for an endoluminal device, the endoluminal device having a flexible first wire disposed therein along a longitudinal direction of the endoluminal device, wherein
the control method comprises:
executing a relaxing operation for relaxing the first wire;
acquiring tension of the first wire during the relaxing operation;
acquiring displacement of the first wire during the relaxing operation; and
adjusting a control parameter of the endoluminal device based on a rate of change in the tension with respect to the displacement during the relaxing operation.

19. The control method according to claim 18, wherein the endoluminal device includes a movable portion, and wherein
the executing the relaxing operation is performed without movement of the movable portion.

20. The control method according to claim 19, wherein the endoluminal device includes a plurality of the first wires, the movable portion is a bending portion, and the plurality of first wires are coupled to the bending portion, and wherein
the control method further includes recognizing a first wire being driven that is being pulled for bending of the bending portion among the plurality of first wires before executing the relaxing operation, and
the executing the relaxing operation without the movement of the movable portion is performed by simultaneously relaxing the first wire being driven and the first wire opposing the first wire being driven.

21. The control method according to claim 19, wherein the endoluminal device includes a plurality of the first wires, the movable portion is a bending portion, and the plurality of first wires are coupled to the bending portion, and wherein
the control method further includes recognizing a first wire being driven that is being pulled for bending of the bending portion among the plurality of first wires before executing the relaxing operation, and
the executing the relaxing operation without the movement of the movable portion is performed by relaxing any of the first wires other than the first wire being driven.
